(19) United States
(12) Patent Application Publication
(10) Pub. No.: US 2010/0260744 A1
(43) Pub. Date: Oct. 14, 2010

(54) REGULATING IL-4 AND IL-13 LEVELS BY BLOCKING HIGH AFFINITY BINDING BY IL-3, IL-5 AND GM-CSF TO THEIR COMMON RECEPTOR

(75) Inventors: Angel Lopz, Adelaide (AU); Paul Foster, Newcastle (AU)

Correspondence Address: FOLEY AND LARDNER LLP;SUITE 500, 3000 K STREET NW, WASHINGTON, DC, 20007, US

(73) Assignees: Medvet Science Pty Ltd.; Newcastle Innovation Ltd.

(21) Appl. No.: 12/668,123

(22) PCT Filed: Jul. 10, 2008
(86) PCT No.: PCT/AU08/01005

§ 371 Date: Jun. 21, 2010

(30) Foreign Application Priority Data
Jul. 11, 2007 (AU) 2007903749

(51) Int. Cl. A61K 39/395; A61P 11/06

(52) U.S. Cl. 424/130.1

(57) ABSTRACT

A method of reducing IL-4 and/or IL-13 levels in the lung of a mammal with elevated levels thereof, includes the step of administering to the mammal an effective amount of a βc receptor blocker capable of blocking the binding of all three of IL-3, IL-5 and GM-CSF to the βc common chain to thereby reduce the IL-4 and/or IL-13 levels.

### FIELD OF THE INVENTION

This invention relates to modulating an immune response connected with an inflammatory condition, most particularly one resulting in reduced IL-4 and IL-13 levels and perhaps other Th2 type cytokines, especially in the lung, as a result of blocking high affinity binding by IL-3, IL-5 and GM-CSF to their common receptor. The invention thus also relates to the treatment, prevention or modulation of inflammatory airways blockage conditions, particularly allergies resulting in conditions such as asthma, and to other allergic conditions and to pharmaceutical compositions therefor.

### BACKGROUND TO THE INVENTION

Two distinct types of T lymphocytes are recognized: CD8⁺ cytotoxic T lymphocytes (CTLs) and CD4⁺ helper T lymphocytes (Th cells).

CTLs recognize and kill cells which display foreign antigens on their surfaces. CTL precursors display T cell receptors that recognize processed peptides derived from foreign proteins, in conjunction with class I MHC molecules, on other cell surfaces. This recognition process triggers the activation, maturation and proliferation of the precursor CTLs, resulting in CTL clones capable of destroying the cells exhibiting the antigens recognized as foreign.

It is now generally accepted that CD4⁺ T cells can be divided into two functionally distinct subsets, T helper 1 (Th1) and T helper 2 (Th2) cells, characterized by the pattern of cytokines which they produce. Thus, mouse Th1 cells produce interferon γ (IFN-γ), tumor necrosis factor β (TNF β), interleukin 2 (IL-2) and interleukin 12 (IL-12), whereas mouse Th2 cells produce IL-3, IL-4, IL-5, IL-6, IL-9, IL-10, and IL-13. Human Th1 and Th2 cells have similar patterns of cytokine secretion, although the synthesis of IL-2, IL-6, IL-10, and IL-13 is not as tightly restricted to a single subset as in the mouse. Several other cytokines are secreted by both Th1 and Th2 cells, including IL-3, TNF α, granulocyte-macrophage colony-stimulating factor (GM-CSF), Met-encephalin, and certain chemokines-(CK).

Th1 and Th2 patterns of cytokine secretion correspond to activated effector phenotypes generated during an immune response. They do not exist among naive T cells. Thus, when first stimulated by antigen on antigen-presenting cells (APC), naive CD4⁺ T cells initially produce only IL-2, and then differentiate into subsets that secrete other cytokines.

Thus Th1 cells are primarily involved in cell mediated immune responses (macrophage activation, antibody-dependent cell cytotoxicity and delayed type hypersensitivity) and resistance to virus infection, and several Th1 cytokines activate cytotoxic and inflammatory reactions. Th2 cytokines potentiate antibody production, particularly IgE responses, and also enhance mucosal immunity through production of growth and differentiation factors for mast cells and eosinophils. Accordingly, Th2 cells are primarily associated with antibody production and allergic reactions.

It is thought that Th1 and Th2 subpopulations arise from a common naive precursor (referred to as ThP). The conditions of antigen stimulation, including the nature and amount of antigen involved, the type of antigen-presenting cells, and the type of hormone and cytokine molecules present seem to all represent determinants of the pattern of Th1 versus Th2 differentiation, with the pivotal role probably, belonging to the cytokines present.

Th1 and Th2 cytokines are mutually inhibitory for the differentiation and effector functions of the reciprocal phenotype. Thus, IL-12 and IFN γ selectively inhibit the proliferation of Th2 cells and IL-4 and IL-10 inhibit Th1 development. Moreover, cytokines produced by Th1 and Th2 antagonize the effector functions of one another.

Th2 cells are believed to produce the cytokines IL-3, IL-4, IL-5, IL-6, IL-9, IL-10, IL-13 and GM-CSF, which are thought to stimulate production of IgE antibodies, as well as be involved with recruitment, proliferation, differentiation, maintenance and survival of eosinophils, which can result in eosinophilia.

The cytokines IL-4 an IL-13 play a key role in the induction of the asthma response not only on account or their pro-inflammatory role, but also due to the effects on mucus hypersecretion and airway wall remodelling (55, 56, 57, 58). IL-4 and IL-13 exhibit overlapping, but not identical effector profiles due to the shared use of the IL-4R α-chain (59). IL-4 and IL-13 have similar effects on B cells, including promoting B-cell proliferation and class switching to IgG4 and IgE. However, unlike IL-4, IL-13 does not appear to be involved in the initial differentiation of CD4 Th2 cells, an important source of pro-inflammatory cytokines, including IL-13. IL-13, on the other hand appears to be more critical to the effector phase of the asthma response, as supported by the observation that IL-13 blockade abolished the asthma phenotype, including airway hyper responsiveness, eosinophil recruitment and mucus overproduction (60).

IL-4 stimulates production of antibodies of the IgE class. IgE is an important component in allergies and asthma. IL-13 is a cytokine that has been implicated in several biological activities including: induction of IgG4 and IgE switching, including in human immature B cells induction of germ line IgE heavy chain (e) transcription and CD23 expression in normal human B cells. Although many activities of IL-13 are similar to those of IL-4 including having a shared common component as its receptor, in contrast to IL-4, IL-13 does not have growth promoting effects on activated T cells or T cell clones.

While the immune system provides tremendous benefits in protecting the body against foreign invaders, particularly those that cause infectious diseases, its effects can be damaging. Thus in the process of eliminating an invading foreign substance some tissue damage may occur, typically as a result of the accumulation of immunoglobulins with non-specific effects. Such damage is generally temporary, ceasing once the foreign invader has been eliminated.

However, there are instances, such as in the case of hypersensitivity or allergic reactions, where the immune response directed against even innocuous agents such as inhaled pollen, inhaled mold spores, insect bite products, medications and even foods, results in severe pathological consequences or symptoms. Many such conditions are thought to involve a pathologic or inappropriate immune response by the humoral branch of the immune system, which is associated with Th2 cell activity.

Diseases involving inflammation are particularly harmful when they afflict the respiratory system, resulting in obstructed breathing, hypoxemia, hypercapnia and lung tissue damage. Obstructive diseases of the airways are characterized by airflow limitation due to constriction of airway smooth muscle, edema and hypersecretion of mucous leading to increased work in breathing, dyspnea, hypoxemia and hypercapnia. While the mechanical properties of the lungs during obstructed breathing are shared between different types of obstructive airway disease, the pathophysiology can differ.

A variety of inflammatory agents can provoke airflow limitation including allergens, cold air, exercise, infections and air pollution. In particular, allergens and other agents in allergic or sensitized mammals cause the release of inflammatory mediators that recruit cells involved in inflammation.

Atopic allergies comprise IgE-mediated diseases in which exposure of an allergic subject to relevant allergens cross-links allergen specific IgE bound to mast cells, triggering degranulation and release of proinflammatory mediators, such as histamine and eicosanoids. Characteristically, this early response is followed by a prolonged late reaction in which inflammatory cells, particularly eosinophils and activated Th2 CD4 T cells, are recruited to the site of allergen exposure. Inflammatory cytokines such as IL-4 and IL-5, both produced by Th2 cells, are important for IgE production by B cells and for eosinophilia, respectively.

IgE is secreted by, and expressed on the surface of B-cells or B-lymphocytes. IgE binds to B-cells (as well as to monocytes, eosinophils and platelets) through its Fc region to a low affinity IgE receptor, known as FcεRII. Upon exposure of a mammal to an allergen, B-cells bearing a surface-bound IgE antibody specific for the antigen are “activated” and develop into IgE-secreting plasma cells. The resulting allergen-specific IgE then circulates through the bloodstream and becomes bound to the surface of mast cells in tissues and basophils in the blood, through the high affinity receptor known as FcεRI. The mast cells and basophils thereby become sensitized for the allergen. Subsequent exposure to the allergen causes a cross linking of basophil and mast cell FcεRI which results in a release of histamine, leukotrienes and platelet activating factors, eosinophil and neutrophil chemotactic factors and the cytokines IL-3, IL-4, IL-5 and GM-CSF which are responsible for clinical hypersensitivity and anaphylaxis.

Although IgEs are produced and released by B-cells, the cells must be activated to do so because B-cells initially-produce only IgD and IgM. The isotype switching of B-cells to produce IgE is a complex process that involves the replacement of certain immunoglobulin constant (C) regions with other C regions that have biologically distinct effector functions, without altering the specificity of the immunoglobulin. This IgE switching is induced in part by IL-4 produced by Th2-cells.

Asthma is a complex and multifactorial disorder, the prevalence of which has increased dramatically in recent decades, particularly in industrialized nations (1-3). Current estimates place the frequency of asthma at 1 in 10 adults and 1 in 4 children in Australia, with similar proportions reported from the UK and USA. Although new approaches for the management and treatment have reduced the mortality rate in recent years (4), the pathophysiological features of asthma are still the basis for significant impact on the quality of life of millions of individuals worldwide.

Clinically, allergic asthma represents an acute or chronic inflammatory disorder characterized by elevated allergen-specific serum IgE, airway eosinophilia, hypersecretion of mucus, airway obstruction, and enhanced bronchial reactivity to nonspecific spasmogenic stimuli (airways hyperreactivity, AHR) (5, 6). The immune response in the asthmatic airway is complex, and the range of inflammatory cells implicated include neutrophils, eosinophils, mast cells, basophils, effector T lymphocytes, and more recently, T regulatory (7-9), natural killer (NK) and NK-T cells (10-13). Importantly, clinical and experimental evidence highlights the obligatory role of aberrant CD4+ T helper 2 (Th2) lymphocyte cytokine responses (e.g., IL-4, IL-5, IL-9, IL-10, and IL-13) to environmental stimuli in the aetiology of disease (14-16). Although clearly a multifactorial syndrome, a prominent feature of allergic asthma is the infiltration of the bronchial tissue and airway lumen by eosinophils (17-19). Within the airway mucosa, the eosinophil has the potential to induce respiratory damage following degranulation and subsequent release of granular proteins, lipid mediators and a range of proinflammatory cytokines and chemokines. Clinically, the presence of these cells and their inflammatory products in the pulmonary compartment often correlates with disease severity (20-23).

Asthma is typically characterized by periodic airflow limitation and/or hyperresponsiveness to various stimuli which results in excessive airways narrowing. Other characteristics can include inflammation of airways, eosinophilia and airway fibrosis.

As with other airway allergies the entire inflammatory process in asthma can also be separated into an early or acute phase and a late or delayed phase. During the acute phase, mast cells degranulate after stimulation and release chemical mediators, including histamines and cytokines. Clinically, this phase is characterized by bronchospasm which can be relieved or prevented by β2 agonists. However, slowly progressive chemical changes involving arachidonic acid begin to occur within mast cells.

Within four to eight hours, a delayed phase occurs as a result of mediator release by inflammatory cells during the initial acute phase of the asthma attack. Eosinophils begin to infiltrate and damage the lower respiratory tract.

Some patients with asthma have very mild symptoms which are easily treated. A significant number of asthmatics however have more severe symptoms and for these individuals currently available treatments such as glucocorticosteroids are ineffective. Chronic asthma is associated with the development of progressive and irreversible airflow reduction due to increasing lung remodelling that results in airway narrowing. Lung remodelling (or airway fibrosis) is the result of fibroproliferative responses to chronic antigen exposure and is correlated with both asthma severity and poor responses to therapy, especially if treatment is delayed. Airway fibrosis due to the deposition of collagen or provisional matrix beneath the basement membrane is often found in asthma patients, even in the airways of patients with mild asthma. Clinical studies have shown a positive correlation between airway fibrosis and airway dysfunction which includes airflow limitation or airways hyperresponsiveness (AHR). The inflammatory mechanisms which result in this collagen deposition are however not fully understood, and reversal of lung remodelling has not been possible.

Currently, therapy for treatment of inflammatory diseases such as moderate to severe asthma predominantly involves the use of glucocorticosteroids. Other anti-inflammatory agents that are used to treat inflammatory diseases include cromolyn and nedocromil. Symptomatic treatment with beta-agonists, anticholinergic agents and methyl xanthines are clinically beneficial for the relief of discomfort, and particularly for early phase reaction but fail to stop the underlying inflammatory processes that cause the disease. None of these treatments inhibit lung remodelling.

The frequently used systemic glucocorticosteroids have numerous side effects, including, but not limited to, weight gain, diabetes, hypertension, osteoporosis, cataracts, atherosclerosis, increased susceptibility to infection, increased lipids and cholesterol, and easy bruising. There is a progressive loss of sensitivity to these treatments after prolonged use, there is limited efficacy of any of these agents in severe cases of asthma, and these agents are non-selective and therefore, side-effects affecting other organs are a potential risk. Furthermore, there are data which document an increased risk of dying from bronchial asthma following prolonged treatment of asthma using long-acting beta-adrenergic agents such as fenoterol. Aerosolized glucocorticosteroids have fewer side effects but can be less potent and have significant side effects, such as thrush.

Other anti-inflammatory agents, such as cromolyn and nedocromil are much less potent and have fewer side effects than glucocorticosteroids. Anti-inflammatory agents that are primarily used as immunosuppressive agents and anti-cancer agents, for example, cytoxan, methotrexate and Immuran have also been used to treat inflammation with mixed results. These agents, however, have serious side effect potential, including, but not limited to, increased susceptibility to infection, liver toxicity, drug-induced lung disease, and bone marrow suppression. Thus, such drugs have found limited clinical use for the treatment of most airway hyperresponsiveness lung diseases.

An alternative to the conventional therapies as outlined above is to take an immunomodulation approach either at the production of IgE antibodies or the imbalance in cytokine profile that is associated with these conditions. In contrast with drug therapy, immunotherapy has the potential to result in long-term, favorable alteration of the patient's immunologic and physiological status.

Current allergy therapies targeting CD4 T cells have met with mixed success. Desensitization with allergen extracts or vaccines is effective for many allergens, such as the Hymenoptera insect sting which can induce life-threatening allergic reactions. The mechanism may be either induction of T cell tolerance or the conversion of Th2 to Th1. However, such treatment requires a long-term treatment regime, frequent doctor visits and prior stabilization by other medications, and is associated with a certain morbidity rate and rare deaths.

Alternative approaches have attempted to use cytokines to shift the immune response. IL-12, a heterodimeric cytokine produced by macrophages and dendritic cells, is potent in driving the development of Th1 cytokine synthesis in naive and memory CD4+ T cells. However, several in vivo studies have demonstrated that rIL-12 as an adjuvant, while enhancing IFN-γ synthesis, in some cases paradoxically also increases IL-4 and IL-10 synthesis in antigen primed CD4⁺ T cells and more relevantly has not been shown to reverse ongoing airway hyperreactivity.

Allergen immunotherapy, while capable of reducing specific IL-4 production, requires multiple injections over several years and is associated with frequent failure.

Trials of immunomodulation approaches have thus far met with limited success and are not yet routinely used as a treatment.

There is now compelling evidence that IL-5, in concert with the chemokine eotaxin, contributes to the maturation and release of eosinophils from hemapoietic progenitors in the bone marrow and the recruitment of this leukocyte to the pulmonary compartment following antigen provocation (24-28). The contribution of IL-5 to allergic asthma was demonstrated by the construction of transgenic mice that constitutively express this cytokine in the lung epithelium. These mice display many of the features of airway disease, such as peribronchial and airway eosinophils, goblet cell hyperplasia, and AHR to methacholine, in the absence of aerosolized allergen challenge (29). However, the effect of IL-5 inactivation on asthma has not been uniformly reported. Although the foremost study demonstrated that the absence of IL-5 in mice with a C57BL/6 genetic background prevents eosinophil accumulation in the lung and AHR (25), this has since been demonstrated to be strain-specific, and BALB/c mice clearly possess an IL-5-independent mechanism of airway disease (30-34).

Further, although anti-IL-5 therapy in mouse models may reduce some aspects of the late-phase asthmatic response, many features of the disease, such as serum IgE and allergen-driven cytokine production, are still present (31). Indeed mice lacking eosinophils have attenuated remodelling in chronic models of asthma.

This disparity is reflected in human trials of anti-IL-5 monoclonal antibodies for treatment of allergic disorders. Although anti-IL-5 therapy was followed by a rapid and sustained decrease in blood eosinophilia in asthmatic patients, the effect on peribronchial eosinophilia was less remarkable (35-38). Further, no significant alterations in airway responsiveness and T cell function have been achieved using this approach (35, 38). Thus, although IL-5 contributes appreciably to pulmonary eosinophilia and modulation of airway function in asthmatics, it has become clear that other mediators may also be of critical importance in regulating eosinophilic inflammation. Appreciation of IL-5 independent pathways of eosinophil function therefore do need to be taken into account.

The cytokines IL-3 and GM-CSF are released at sites of allergic inflammation and together with IL-5, are recognised as being the only mediators capable of inducing eosinophil production and promoting maturation, activation, migration and survival of this cell type, both in vitro and in vivo (14, 39-41). The action of these cytokines on eosinophils is mediated by specific receptor heterodimers. IL-3, IL-5 and GM-CSF each possesses a unique a receptor subunit (IL-3Rα, IL-5Rα and GM-CSFRα) that binds specifically to its ligand and upon binding engages a common β receptor subunit (βc). This interaction provides the requisite spatial and conformational arrangement of the α and B subunits to initiate physiological effects through diverse signalling mechanisms including the JAK/STAT pathway, the MAPK pathway and the PI3-K cascade (42-45). From a therapeutic perspective, functional inactivation of the βc receptor subunit would allow antagonism of all three eosinophilopoietic cytokines using a single agent and thus has the potential to eliminate many of the pathophysiological features of asthma. It should be noted that unlike in the human, the murine system encompasses an additional B receptor specific for IL-3 (β_{IL-3}), which is highly homologous to βc and able to form a functional complex with IL-3 and its α subunit, facilitating some residual IL-3 signalling independent of βc.

The importance of βc in eosinophil biology has been highlighted by both in vitro and in vivo studies. Mice null for the βc receptor show reduced numbers of eosinophils in the bone marrow and peripheral blood at baseline conditions, in the absence of any other haematological abnormalities (46). Further, bone marrow cells from βc−/− mice in the study failed to respond to IL-5 and GM-CSF in clonal cultures. An independent study by Nishinakamura and co-workers confirmed the low basal number of eosinophils in βc null mice and demonstrated that in the absence of this receptor the immune response to infection by the parasite Nippostronglus brasiliensis is abrogated, characterised by an absence of eosinophilia in the blood and lung (47). In the human, functional inactivation of (βc on purified eosinophils by the monoclonal antibody BION-1 blocks the high affinity binding of IL-3, IL-5 and GM-CSF and subsequent receptor activation by preventing heterodimerisation and βc phosphorylation (48).

Although the requirement for βc for eosinophil function at baseline and during parasite infection has been explored, the role of this receptor in allergic inflammation has not yet been fully appreciated. Specific blocking of binding of Lyn kinase to βc using a stearated peptide inhibitor prevented eosinophil differentiation from the stem cell pool and cell survival, however did not influence eosinophil degranulation and mediator release (49). Although this was sufficient to reduce pulmonary eosinophil numbers in a murine model of asthma, this granulocyte was still a major feature of the airway in treated mice, and no impact on other pathophysiological features of asthma was demonstrated. The only report addressing the impact of specifically targeting βc on allergic inflammation originates from Allakhverdi and co-workers, who employed antisense oligonucleotide inhibitors directed against the common β chain in a rat model of allergic airways disease (50). They observed a reduction in airway eosinophilia and airway hyperresponsiveness, supporting the concept of the βc as a therapeutic target. However, receptor expression was only reduced by 60%, and although significantly abrogated, airway parameters were still notably higher than baseline levels in nonallergic mice. Notably, the aforementioned studies employ mice null for the βc gene, but with intact IL-3 responses via the murine β_{IL-3} receptor. No reports of the immune response in mice lacking both β receptor molecules (βc/β_{IL-3} double knockout) and therefore fully IL-3 immunoincompetent have been made.

There has been no indication in the prior art that a reduction of signalling from βc can effect a reduction in IL-4 and IL-13, and consequent inhibition of the Th2 type response, nor any indication that blocking of βc mediated signalling would provide a significant impact on lung remodelling.

### SUMMARY OF THE INVENTION

This invention arises from the finding that blocking of common receptor βc mediated signalling of all three of IL-3, IL-5 and GM-CSF leads to a reduction in IL-4 and IL-13 levels in inflammatory conditions particularly in the lung. This reduction is suggested to result in a significant shift from Th2 immune reaction to a Th1 immune reaction, and a range of markers tested bears out that such a shift has occurred. The impact of such a shift has been investigated in a mouse asthma model and is found to alleviate a number of symptoms and markers associated with asthma, furthermore a significant effect on lung remodelling is shown in this obstructive airways condition model.

Thus the invention in a first aspect could be said to reside in a method of reducing IL-4 and/or IL-13 levels in the lung of a mammal with elevated levels thereof, including the step of administering to the mammal an effective amount of a βc receptor blocker capable of blocking the binding of all three of IL-3, IL-5 and GM-CSF to the βc common chain in said mammal thereby reducing the IL-4 and/or IL-13 levels.

In a second aspect the invention may be said to reside in a method of inhibition or reversing lung remodelling in a patient with an obstructive airways condition, the method including the step of administering an effective amount of a βc blocker capable of blocking the binding of all three of IL-3, IL-5 and GM-CSF to the common βc receptor; said βc blocker being administered for a time sufficient to cause a lung remodelling effect.

In a third aspect the invention could be said to reside in a method of treatment of a severe obstructive airway condition in a mammal, the condition being refractory to treatment with glucocorticosteroids, the method comprising the step of administering an effective amount of βc blocker capable of blocking the binding of all three of IL-3, IL-5 and GM-CSF to the common βc.

In a fourth aspect the invention could be said to reside in a method of prescribing treatment for airway hyper-responsiveness and/or airflow limitation associated with a respiratory condition involving an inflammatory response in a mammal, comprising:
a. administering to the mammal a βc blocker capable of blocking the binding of all three of IL-3, IL-5 and GM-CSF to the common βc receptor,
b. measuring a change in respiratory function in response to a provoking agent in said mammal to determine if said βc regulating agent modulates airway hyperresponsiveness; and
c. prescribing a pharmacological therapy comprising administering a dose of the βc blocker to the mammal effective to reduce inflammation based upon said changes in lung function.

In a fifth aspect the invention could be said to reside in a method of biasing an immune response away from a Th2 immune response by administering a βc blocker capable of blocking the binding of all three of IL-3, IL-5 and GM-CSF to their common βc receptor to thereby change levels of one or more markers indicative of a Th2 response.

In a sixth aspect the invention could be said to reside in a method of converting an established antigen-specific allergic response characterized by the production of Th2-type cytokines to a Th1-type response, the method comprising administering an effective dose of antigen in conjunction with a βc blocker for a period of time sufficient to convert said antigen-specific allergic response to a Th1-type response, the βc blocker capable of blocking the binding of all three of IL-3, IL-5 and GM-CSF to their common βc receptor to thereby change levels of one or more markers indicative of a Th2 response.

In a seventh aspect the invention could be said to reside in a method of treating asthma associated allergies, the method comprising:
administering to a patient an effective dose of an asthma associated allergen in conjunction with a βc blocker, the βc blocker capable of blocking the binding of all three of IL-3, IL-5 and GM-CSF to their common βc receptor;
wherein the effects of the asthma associated allergies are decreased.

In an eighth aspect the invention could be said to reside in a composition comprising a βc blocker and an allergen the subject of an antigen specific response and a pharmaceutically acceptable carrier.

In a ninth aspect the invention could be said to reside in a composition for non-pulmonary delivery, comprising a βc blocker and a pharmaceutically acceptable carrier, preferably being a controlled release composition.

In a tenth aspect the invention could be said to reside in a medicament for use in reducing IL-4 and/or IL-3 levels said medicament when administered being capable of blocking the binding of all three of IL-3, IL-5 and GM-CSF to their common βc receptor thereby reducing IL-4 and IL-13 levels. It will also be understood that the invention may relate to a method of making a medicament according to the tenth aspect of the invention.

### BRIEF DESCRIPTION OF THE DRAWINGS

FIG. 1: Characterization of inflammatory cell infiltrates in bronchoalveolar lavage fluid (BALF) from wild-type (WT) and βc−/− mice. Lungs were flushed 24 h after the final OVA challenge and differential counts performed on May-Grunwald Giemsa stained-cytospins. Data represents the mean±SEM for a minimum of 6 mice per group for A. Neutrophils. * p<0.05 compared to naïve, **p<0.005 compared to naïve, # p<0.005 compared to nonallergic. B. Lymphocytes. * p<0.05 compared to naïve, # p<0.05 compared to nonallergic. C. Macrophages. * p<0.05 compared to naïve, ** p<0.001 compared to naïve and allergic. D. Eosinophils. FIG. 2: Histological examination of pulmonary tissue in wild-type (WT) and βc−/− mice. A. Formalin-fixed lungs were sectioned and stained with Carbol's-chromatrope haematoxylin for eosinophil determinations. Peribronchial eosinophils in 10 similar high powered fields within 100 μm of the basement membrane were counted for each lung. B. Formalin-fixed lungs were sectioned and stained with alcian blue/periodic acid-Schiff for enumeration of mucus-secreting cells (MSC) in the bronchial epithelium. 10 similar high powered fields within 100 μm of the basement membrane were counted for each lung. Data represents mean±SEM for a minimum of 6 mice per group. *** p<0.001, ** p<0.005 and * p<0.05 compared to naïve and nonallergic mice for that strain. Levels of significant differences are indicated for other groups.

FIG. 3: Measurement of airways hyperreactivity in wild-type (WT) and βc −/− mice. Airway reactivity to inhaled methacholine was measured 24 h after the final aeroallergen challenge. A. Airway resistance (R_{L}) and B. Dynamic compliance (C_{Dyn}) are represented as a percentage of the baseline reactivity to saline in the absence of cholinergic stimuli and represent the mean±SEM for a minimum of 6 mice per group. The maximal dose to methacholine (25 mg/ml) is shown, but this is representative of the full dose response curve. * p<0.001 between respective nonallergic and allergic groups. Levels of significant differences are indicated for other groups.

FIG. 4: Peribronchial lymph node (PBLN) antigen-specific proliferation and cytokine production in wild-type (WT) and βc −/− mice. A. Splenocyte and PBLN cells from OVA sensitised and challenged (allergic) mice were cultured for 3 days in the presence of OVA. Unstimulated cells were cultured in media only. Proliferation was determined using the CellTiter reagent and expressed as the percentage increase over the corresponding unstimulated control. Data represents mean±SEM for a minimum of 6 replicate cultures. ** p<0.01 compared to WT, * p<0.05 compared to WT. B-D. PBLN cells were isolated from allergic and nonallergic mice and cultured for 6 days in the presence of OVA. Supernatants were collected and IL-5, IL-13, IL-4 and IFN-γ measured by ELISA. Data represents mean±SEM for a minimum of 6 replicate cultures. ** p<0.001 and * p<0.05 compared to nonallergic mice for that strain. Levels of significant differences are indicated for other groups.

FIG. 5: Serum OVA-specific immunoglobulins in allergic wild-type (WT) and βc−/− mice. Antigen-specific IgE (A), IgG₁ (B) and IgG₂ₐ (C) in serum from OVA sensitised and challenged mice were measured by ELISA. Data represents mean±SEM for a minimum of 6 mice. No OVA-Ig were detected in naïve and nonallergic mice (data not shown). * p<0.001 compared to WT.

FIG. 6: Temporal analysis of eosinophilic infiltration in allergic wild-type (WT) and βc −/− mice. Mice were sampled at various time points after the final OVA challenge and eosinophilia were counted in May-Grunwald Giemsa stained blood smears (A), bronchoalveolar lavage fluid (BALF) cytospins (B), and Carbol's-chromatrope haematoxylin stained lung tissue (C). Data represents the mean±SEM for a minimum of 5 mice per group. * p<0.05 and *p<0.001 compared to corresponding time point in WT strain.

FIG. 7: In vitro Th2 polarisation of CD4+ T cells from naïve wild-type (WT) and βc −/− mice. CD4+ T cells were purified from the spleens of naïve mice and cultured for 4 d under conditions designed to promote Th2 differentiation. Cells were then washed and cultured for a further 6 d with anti-CD3 and anti-CD28 alone, after which supernatants were collected and cytokines measured by ELISA. (A) IL-5, (B) IL-13, (C) IL-4, (D) IFNγ and (E) GM-CSF. Data represents the mean±SEM for a minimum of 5 mice per group. ** p<0.0001 and * p<0.01 compared to unstimulated cultures. Levels of significant differences between strains are indicated.

FIG. 8: Lymphocyte profile in peribronchial lymph node (PBLN) and lung homogenates from wild-type (WT) and βc −/− mice. PBLN and lung cells were isolated from nonallergic and allergic mice, stained with surface markers and analysed by flow cytometry. Lymphocyte profiles were analysed in (A) PBLN and (C) lungs of allergic mice. * p<0.05 and ** p<0.001 compared to WT. The cell surface marker CD69 was used to assess cell activation in (B) PBLN and (D) lungs of nonallergic and allergic mice. * p<0.05 and ** p<0.001 compared to WT. Differences between treatment groups are indicated.

FIG. 9: Dendritic cell profile in peribronchial lymph node (PBLN) and lung homogenates from wild-type (WT) and βc−/− mice. PBLN and lung cells were isolated from nonallergic and allergic mice, stained with surface markers and analysed by flow cytometry. Numbers of myeloid dendritic cells (mDCs; CD11c+CD11b+) and plasmacytoid dendritic cells (pDCs; CD11c+CD11b−GR1+PDCA1+) were analysed in (A) PBLN and (C) lungs of allergic mice. * p<0.005 and ** p<0.001 compared to WT. Activation of myeloid dendritic cells was explored in nonallergic and allergic mice by staining for costimulatory molecules MHCII, CD80 and CD86 in (B) PBLN and (D) lung preparations. * p<0.05, ** p<0.001 compared to nonallergic mice of the same strain. Differences between strains are indicated.

### DETAILED DESCRIPTION OF THE INVENTION

A first aspect of the invention resides in a method of reducing IL-4 and/or IL-13 levels in the lung of a mammal with elevated levels thereof, including the step of administering to the mammal an effective amount of a βc receptor blocker capable of blocking the binding of all three of IL-3, IL-5 and GM-CSF to the βc common chain in said mammal to reduce IL-4 and or IL-13 levels in the lung.

Blocking of βc signalling in experiments conducted thus far has led to a reduction in both of IL-4 and IL-13 and it is believed that blocking βc signalling will lead to a reduction in both. It is possible however that there may be a differential reduction so that there is significant reduction in just one or other of these two cytokines which at one extreme would provide for no reduction in one of them. It is postulated that this will still have a beneficial effect because elevated levels of each of these cytokines are important effectors leading to the adverse reaction in allergies. Blocking of both however is likely to provide for greater alleviation of conditions arising from or contributed to by such elevated levels

The βc blocker blocks binding of all three of IL-3, IL-5 and GM-CSF to the βc common receptor, and therefore blocks βc common receptor mediated signalling. The blocking of signalling resulting from binding by all three of these cytokines provides the beneficial effect. It has been found (47) that blocking of GM-CSF and IL-5 only leads to a residual, albeit delayed, signal that provides for adverse effects associated therewith. It is accordingly desirable to block binding of all three of the cytokines that signal via the βc common receptor.

The blocking contemplated by this invention may be a full blockage thus the cytokines GM-CSF, IL-3 and IL-5 are completely prevented from binding the common receptor, but may also be a partial blockage of all three cytokines that is of sufficient magnitude to provide a beneficial effect.

The blocking may be for a short period, such as for example where an acute attack, for example of asthma, is to be treated. It is expected that blocking all βc signalling over a prolonged time throughout all of the mammal is likely to lead to undesirable side effects such as pulmonary alveolar proteinosis, reduction in monocyte and dendritic function. Accordingly it is desired that the blocking, and therefore reduction in IL-4 and/or IL-13 is temporally limited. It may be desired that blocking of (βc signalling is repeated so that a βc blocker might be administered two or more times temporally spaced apart.

One surprising finding of the present inventors is that the effects of βc blocking in the manner set out in the examples below does not lead to reduction of IL-4 and IL-13 throughout the mammal, rather there was a differential effect, a reduction being found in the peribronchial lymph nodes (PBLN) draining the respiratory tract, whereas no reduction was found in the spleen. This preferential reduction in lung IL-4 and/or IL-13 levels relative to systemic IL-4 and/or IL-13 levels has benefits where a lung condition associated with elevated IL-4 and/or IL-13 is to be treated. Thus the use of a βc blocker is ideally suited to target an inflammatory obstructive airways condition. This has two consequences, the first being that it is likely that adverse side effects of blocking βc signalling will not be as severe over an extended period because a less significant or no whole of body reduction in the two cytokines is required to still have a therapeutic effect in the pulmonary system. Accordingly approaches to treatment that provide for extended delivery of the βc blocker are likely to have less side effects and therefore are a practical approach. These extended exposure strategies include, for example, reversal of an adverse immune response. Such approaches might include the slow or controlled release of a βc blocker, rather than the application of a single or multiple discrete doses.

Such controlled release approaches might include delivery of a pharmaceutical composition by way of a dermal patch or other depot perhaps introduced into other body locations, slow release oral compositions, or alternatively where the βc blocker is a protein or nucleic acid by gene therapy methods.

Another corollary of the finding of preferential lung effect on IL-4, and IL-13 in conditions associated with the lung, is that there is less benefit in attempting to specifically target delivery of the βc blocker to the lung. Thus it is anticipated non-pulmonary delivery is likely to be as effective as pulmonary delivery. This has its benefits if only because such non-pulmonary delivery is more readily accepted by a patient, and compositions are in general more readily formulated. Thus for example a dermal patch is a relatively unintrusive approach, and is more readily formulated for slow release and the same applies to an oral formulation.

Forms of βc blockers are set out in earlier US patent specifications 6200567 (also patent publication WO97/28190) which refers to the F′-G′ loop of domain 4 and certain amino acids of the loop as playing a critical role for the high affinity binding of all three of IL-3, IL-5 and GM-CSF to the common βc receptor. US patent specification 6720155 refers to monoclonal antibodies including BION-1 as binding to both the B′-C′ loop and the F′-G′ loop of domain 4 of the common βc receptor. As a result of the binding βc mediated signalling is blocked. The βc blocker of the present invention includes the forms set out in the two US patent specifications referred to above which are incorporated herein in their entirety.

Thus βc blockers may include any pharmaceutically acceptable molecule that blocks the binding of all three of IL-3, IL-5 and GM-CSF to their common βc receptor, and thus may include molecules that bind to common βc receptors to thereby block binding of the three cytokines or a molecule that provides a βc mimic of the common βc receptor but that does not result in βc mediated signalling. The latter may be provided to competitively bind the three cytokines and can include a modified βc receptor or more preferably a fragment or mimetope thereof. The fragment may include all or part of domain 4 wherein the F′-G′ loop and or the B′-C′ loop remain in a configuration to bind the three cytokines. This may be administered as a polypeptide perhaps stabilised to prevent degradation on administration by known methods. Alternatively this may be administered as a nucleic acid encoding the βc receptor mimic delivered to express the βc mimic. This is preferably administered to the lung perhaps carried on a non-replicable lentivirus vector or other suitably approved safe vector.

The molecules that bind to common βc receptor may take the form of any one of a number of classes of compounds and may be selected from a group comprising, antibodies or fragments thereof, peptides, oligosaccharides, oligonucleotides, or other organic or inorganic compounds.

As indicated above, a βc blocker of the present invention can be any agent that blocks the binding of all three of IL-3, IL-5 and GM-CSF to their common βc receptor. Additionally, a βc blocker of the present invention can include the common βc receptor or fragments thereof that bind all three cytokines but does not lead to signalling, in the form of either an isolated protein (as an exogenous protein) or an isolated nucleic acid molecule encoding the common βc receptor or fragments thereof.

βc blockers include, for example, compounds that are products of rational drug design, natural products, and compounds having partially defined βc blocking properties. A βc blocker can be a protein-based compound, a carbohydrate-based compound, a lipid-based compound, a nucleic acid-based compound, a natural organic compound, a synthetically derived organic compound, an antibody, or fragments thereof.

A βc blocker can be obtained, for example, from molecular diversity strategies (a combination of related strategies allowing the rapid construction of large, chemically diverse molecule libraries), libraries of natural or synthetic compounds, in particular from chemical or combinatorial libraries or by rational drug design. See for example, Maulik et al., 1997, Molecular Biotechnology: Therapeutic Applications and Strategies, Wiley-Liss, Inc., which is incorporated herein by reference in its entirety.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands against a desired target, and then optimize the lead molecules by either random or directed design strategies. Methods of molecular diversity are described in detail in Maulik, et al., ibid.

In a rational drug design procedure, the three-dimensional structure of a regulatory compound can be analyzed by, for example, nuclear magnetic resonance (NMR) or X-ray crystallography. In the case of the βc common receptor this three dimensional structure has been published (61). This three-dimensional structure can be used to predict structures of potential compounds, such as potential βc blockers by, for example, computer modelling. The predicted compound structure can be used to optimize lead compounds derived, for example, by molecular diversity methods. In addition, the predicted compound structure can be produced by, for example, chemical synthesis, recombinant DNA technology, or by isolating a mimetope from a natural source (e.g., plants, animals, bacteria and fungi).

A βc blocker which is an antibody can be an antibody which selectively binds to the F′-G′ and/or B′-C′ loop of domain 4 common βc receptor or mimetope thereof or adjacent the two loops such as to block high affinity binding of all three of IL-3, IL-5 and GM-CSF thereto. Such an antibody can be referred to herein as a βc blocker antibody. βc blocker antibodies can selectively bind to the common βc receptor. As used herein, the term “selectively binds to” refers to the ability of such an antibody to preferentially bind to common βc receptor. Antibodies useful in the present invention can be either polyclonal or monoclonal antibodies. Such antibodies can include, but are not limited to, neutralizing antibodies, non-neutralizing antibodies, and complement fixing antibodies. Antibodies useful in the present invention include functional equivalents such as antibody fragments and genetically-engineered antibodies, including single chain antibodies, that are capable of selectively binding to at least one of the epitopes of the protein or mimetope used to obtain the antibodies. Antibodies useful in the present invention can include chimeric antibodies in which at least a portion of the heavy chain and/or light chain of an antibody is replaced with a corresponding portion from a different antibody. For example, a chimeric antibody of the present invention can include an antibody having an altered heavy chain constant region, an antibody having protein sequences derived from two or more different species of mammal, and an antibody having altered heavy and/or light chain variable regions.

In aspects of the invention a βc blocker is used in a pharmaceutical composition. While it is possible to administer the βc blocker on its own, it is preferred to be presented as part of a pharmaceutical composition. In accordance with this aspect of the invention, the pharmaceutical composition comprises a βc blocker in a therapeutically effective dose together with one or more pharmaceutically acceptable carriers and optionally other therapeutic ingredients. A wide variety of pharmaceutically acceptable carriers are known. See, for example Remington's Pharmaceutical Sciences, Mack Publishing Co., Easton, Pa., (1990), which is incorporated by reference herein. Preferred carriers include inert, non-toxic solids, (commonly used non toxic solids include dextrose, dextrin, cellulose, pectin, starch, lactose, sucrose, and calcium phosphate,), semi-solids (commonly used semi-solids include glycerol stearate, polyethylene glycol, stearic acid, agar, gelatin, and propylene glycol) and liquids (commonly used liquids include buffered saline, water, an organic solvent, and pharmaceutically acceptable oils or fats).

The preferred form of the composition of (βc blocker will depend on the intended mode of administration, which in turn will depend on the location and nature of the inflammatory disorder to be treated. For example, delivery to the mouth, head and/or neck can be in the form of aqueous-based oral solutions, suspensions, emulsions, syrups, elixirs, gels, patches, lozenges, tablets, or capsules. Delivery to the gastrointestinal tract can be in the form of oral solutions, gels, suspensions, tablets, capsules, and the like. It is also possible to formulate the βc blocker preparation for rectal administration in the form of an enema, suppositories, rectal-foam, and the like. Delivery to the eye can be in the form of solutions, gels, or suspensions. Delivery to the nose can be in the form of solutions, gels, or suspensions. The intranasal formulations may be formulated, for example, into an aqueous or partially aqueous solution, which can then be utilized in the form of a nasal drop or an aerosol. Delivery to the skin can be in the form of aqueous-based solutions, gels, suspensions, lotions, creams, ointments, patches, and the like.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral administration include water (partially containing additives as above), alcohols (including monohydric alcohols and polyhydric alcohols) and their derivatives, oils (for example peanut oil, sesame oil, olive oil, and coconut oil), and combinations of the above. Compositions comprising such carriers and adjuvants may be formulated using well known conventional materials and methods. Such materials and methods are described, for example, in Remington's Pharmaceutical Sciences, supra.

A solid carrier can include one or more substances which may also act as flavoring agents, lubricants, solubilizers, suspending agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablet preferably contain up to 99% of the active ingredient, and may be formulated for immediate and/or sustained release of the active ingredient. Suitable solid carriers include, for example, calcium or sodium phosphate, magnesium stearate, talc, sugars, glycine, lactose, dextrin, starch, gelatin, cellulose, cellulose derivatives (for example methyl cellulose, hydroxypropylmethyl cellulose, and sodium carboxymethyl cellulose), polyvinylpyrrolidone, low melting point waxes, and combinations of the above.

Oral tablets may be prepared using a variety of well known methods and in a variety of conventional forms. Exemplary forms include dry powder compaction tablets, micro-particulate systems (for example wherein the active ingredient is spray-dried onto a scaffold particle), and hard or soft-gel capsules. The tablets may be optionally covered with an enteric coating, which remains intact in the stomach, but will dissolve and release the contents of the tablet once it reaches the small intestine. Most currently used enteric coatings are those which remain undissociated in the low pH environment of the stomach, but readily solubilize when the pH rises to about 4 or 5. A number of commercially available enteric coatings may be used depending on the target part of the intestinal tract. Such coatings include, for example, methacrylic acid-methacrylic acid ester-based copolymer, which is sold under the trade name “Eudragit”; anionic water-soluble, polymer cellulose ether, which is sold under the trade name “Aqualon”; cellulose acetate phthalate; polyvinyl acetate phthalate; hydroxypropyl methylcellulose phthalate; and the like. Compositions comprising such carriers and adjuvants may be formulated, and tablets prepared from such compositions, using well known conventional materials and methods. Such materials and methods are described, for example, in Remington's Pharmaceutical Sciences, supra.

For administration by inhalation, the βc blocker is conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of for example gelatin for use in an inhaler or insufflator can be formulated containing a powder mix of the compound and a suitable powder base such as lactose or starch.

In one embodiment of the invention, the pharmaceutical composition comprises one or more sustained or controlled release excipients such that a slow or sustained release of the active ingredient is achieved. A wide variety of suitable excipients are known.

Compositions including the βc blocker may also be formulated as a depot preparation. Such long acting formulations may be administered by implantation (for example subcutaneously or intramuscularly) or by intramuscular injection. Thus, for example, the modulating agents may be formulated with suitable polymeric or hydrophobic materials (for example as an emulsion in an acceptable oil) or ion exchange resins, or as sparingly soluble derivatives, for example, as a sparingly soluble salt.

Administration can also be by transmucosal or transdermal means. For transmucosal or transdermal administration, penetrants appropriate to the barrier to be permeated are used in the formulation. Such penetrants are generally known, and include, for example, for transmucosal administration bile salts and fusidic acid derivatives. In addition, detergents may be used to facilitate permeation. Transmucosal administration may be through nasal sprays or using suppositories. For topical administration, the β_{c} blockers of the invention are formulated into ointments, salves, gels, or creams as generally known, and for slow release dermal patches.

The βc blocker either alone or in combination with other therapeutic agents, may also be administered topically in the form of a dermal patch or transdermal delivery system. In this embodiment of the invention, the pharmaceutical composition may be administered through the use of a dermal patch containing the active ingredient(s) and a carrier that is inert to the active ingredient(s), non-toxic to the skin or mucosal epithelium, and allows delivery of the agent to the dermis and/or epithelium. Dermal patches and delivery systems, utilizing active or passive transdermal delivery carriers, comprising βc blocker may be prepared using well known methods and materials, including, for example, microporous membranes, silicon polymers and diffusion matrixes. Such materials and methods are described, for example, in Remington's Pharmaceutical Sciences, supra.

Further guidance in preparing pharmaceutical formulations can be found in, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al., (eds), 1993, Pharmaceutical Dosage Forms Parenteral Medications, Dekker, N.Y.; Lieberman et al. (eds), 1990, Pharmaceutical Dosage Forms: Disperse Systems, Dekker, N.Y.

The subject modulating agents can be administered to a subject at therapeutically effective doses to treat or ameliorate a disorder benefiting from the βc blocker. The data obtained from cell culture assays and animal studies can be used in formulating a range of dosages for use in humans. The dosage of such modulating agents lies preferably within a range of circulating or tissue concentrations that include the ED50 with little or no toxicity. The dosage may vary within this range depending upon the dosage form employed and the route of administration utilized. For any modulating agent used in the method of the invention, the therapeutically effective dose can be estimated initially from cell culture assays. A dose may be formulated in animal models to achieve a circulating plasma concentration range that includes the IC50 (that is, the concentration of the test modulating agent which achieves a half-maximal inhibition of symptoms) as determined in cell culture. Such information can be used to more accurately determine useful doses in humans. Levels in plasma may be measured, for example, by high performance liquid chromatography.

Preferably the amount of βc blocker administered is sufficient to treat, prevent or modulate a condition or disease. This refers to reducing the potential for an inflammatory response and the effectiveness of which can be tested against one or more provoking agents, for example, methacholine, histamine, an allergen, a leukotriene, saline, hyperventilation, exercise, sulfur dioxide, adenosine, propranolol, cold air, antigen and bradykinin. Preferably, the potential for an inflammatory response is reduced, optimally, to an extent that the mammal no longer suffers discomfort and/or altered function from exposure to the inflammatory agent. For example, treating or protecting a mammal can refer to the ability of a compound, when administered to the mammal, to prevent a disease from occurring and/or cure or alleviate disease symptoms, signs or causes. In particular, protecting a mammal refers to modulating an inflammatory response to suppress an overactive or harmful inflammatory response. Also in particular, protecting a mammal refers to regulating cell-mediated immunity and/or humoral immunity. Treating protecting or modulating a mammal can also refer to a reduction or prevention of symptoms associated with the disease, such as a reduction or prevention of airways fibrosis.

In addition, the invention may contemplate using gene therapy for treating a mammal, using nucleic acid encoding βc common receptor antagonist, if it is a protein. Generally, gene therapy is used to over express βc common receptor antagonist levels in the mammal. Nucleic acids which encode the βc common receptor antagonist, under suitable regulatory control can be used for this purpose or expression of non signalling βc common receptor.

There are two major approaches to getting the nucleic acid (optionally contained in a vector) into the patient's cells for purposes of gene therapy: in vivo and ex vivo. For in vivo delivery, the nucleic acid is injected directly into the patient, usually at the site where βc common receptor mimic is required, in the present invention this is preferably to the lung. For ex vivo treatment, the patient's cells are removed, the nucleic acid is introduced into these isolated cells and the modified cells are administered to the patient either directly or, for example, encapsulated within porous membranes which are implanted into the patient. See, e.g. U.S. Pat. Nos. 4,892,538 and 5,283,187.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus. Many types of cells and cell lines (e.g., primary cell lines or established cell lines) and tissues are capable of being stably transfected by or receiving the constructs of the invention. Examples of cells that may be used include, but are not limited to, stem cells, B lymphocytes, T lymphocytes, macrophages, other white blood lymphocytes (e.g., myelocytes, macrophages, or monocytes), immune system cells of different developmental stages, erythroid lineage cells, pancreatic cells, lung cells, muscle cells, liver cells, fat cells, neuronal cells, glial cells, other brain cells, transformed cells of various cell lineages corresponding to normal cell counterparts (e.g., K562, HEL, HL60, and MEL cells), and established or otherwise transformed cell lines derived from all of the foregoing. In addition, the constructs of the present invention may be transferred by various means directly into tissues, where they would stably integrate into the cells comprising the tissues. Further, the constructs containing the DNA sequences of the peptides of the invention can be introduced into primary cells at various stages of development, including the embryonic and fetal stages, so as to effect gene therapy at early stages of development.

In vivo nucleic acid transfer techniques include transfection with viral vectors (such as adenovirus, Herpes simplex I virus, adeno-associated virus, or lentivirus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, and the like. Where liposomes are employed, proteins which bind to a cell surface membrane protein associated with endocytosis may be used for targeting and/or to facilitate uptake, for example, capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414 (1990). For review of known gene marking and gene therapy protocols, see Anderson et al., Science, 256: 808-813 (1992). See also WO 93/25673 and the references cited therein. Other discussions of how to perform gene therapy in a variety of cells using retroviral vectors can be found, for example, in U.S. Pat. No. 4,868,116, issued Sep. 19, 1989, and U.S. Pat. No. 4,980,286, issued Dec. 25, 1990 (epithelial cells), WO89/07136 published Aug. 10, 1989 (hepatocyte cells), EP 378,576 published Jul. 25, 1990 (fibroblast cells), and WO89/05345 published Jun. 15, 1989 and WO/90/06997, published Jun. 28, 1990 (endothelial cells), the disclosures of which are incorporated herein by reference.

The invention has particular benefit where the mammal has an obstructive airway condition and in particular wherein the condition is allergic and/or inflammatory. “Obstructive airways condition” includes clinical and subclinical conditions and includes “disease” which includes an apparent inflammatory manifestation, whereas subclinical may only manifest in a reduced lung function and may only be seen fully in histological analysis of biopsies. An “obstructive lung disease” or “obstructive airway disease” (OAD) are terms used to describe a complex of chronic and acute conditions that have in common airflow limitation or airflow obstruction. The sites of airway obstruction in OADs vary from the upper airways to the most peripheral bronchioles. OADs sufferers all have airway narrowing as a disease parameter and they also share inflammation as a component of the disease process. Such airway obstruction is usually caused by infiltration of inflammatory cells, scarring, edema, smooth muscle hypertrophy/hyperplasia, smooth muscle contraction and narrowing due to secretions typically mucous. Such conditions include asthma, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma (that is, asthma, wheezing, chest tightness and cough caused by a sensitizing agent, such as an allergen, irritant or hapten, in the work place), sarcoid, reactive airway disease syndrome (that is, a single exposure to an agent that leads to asthma), interstitial lung disease, hyper-eosinophilic syndrome, rhinitis, sinusitis, or parasitic lung disease. The present invention is particularly applicable to asthma, emphysema, chronic bronchitis, and chronic bronchiolitis. An obstructive airways condition may be any of these or subclinical manifestations of these.

The invention may also relate to diseases that are thought to be caused/mediated in substantial part by a Th2 immune response, IL-4/IL-5 cytokine induction, and/or eosinophilia and accordingly are responsive to treatment by administering a therapeutically effective amount of a βc blocker. Such conditions include asthma, allergic rhinitis, systemic lupus erythematosis, Ommen's syndrome (hypereosinophilia syndrome), certain parasitic infections, for example, cutaneous and systemic leishmaniasis, toxoplasma infection and trypanosome infection, and certain fungal infections, for, example candidiasis and histoplasmosis, and certain intracellular bacterial infections, such as leprosy and tuberculosis. These are examples of non-viral and non-tumor, Th2 mediated diseases. The invention may be applicable also to subclinical manifestations of these diseases. Particularly preferred uses of the present invention are for the treatment of diseases associated with eosinophilia, such as asthma and allergic rhinitis.

Yet other aspects of the invention may be applicable to treatment, prevention or modulation of diseases associated with, or mediated or caused by, IgE production and/or accumulation that may be treated or prevented according to the methods of the invention include, but are not limited to anaphylactic hypersensitivity or allergic reactions and/or symptoms associated with such reactions (including food and drug allergies), allergic rhinitis, allergic conjunctivitis, systemic mastocytosis, hyper IgE syndrome, and IgE gammopathies, atopic disorders such as atopic dermatitis, atopic eczema and atopic asthma, and B-cell lymphoma

The first and other aspects of the invention may relate to treatment, prevention or modulation of various medical conditions in which IL-13 is implicated or which are effected by the activity (or lack thereof) of IL-13 (collectively “IL-13-related conditions”). IL-13-related conditions include without limitation Ig-mediated conditions and diseases, particularly IgE-mediated conditions (including without limitation allergic conditions, asthma, immune complex diseases (such as, for example. lupus, nephrotic syndrome, nephritis, glomerulonephritis, thyroiditis and Grave's disease)); immune deficiencies, specifically deficiencies in hematopoietic progenitor cells, or disorders relating thereto; cancer and other disease. Such pathological states may result from disease, exposure to radiation or drugs, and include, for example, leukopenia, bacterial and viral infections, anemia, B cell or T cell deficiencies such as immune cell or hematopoietic cell deficiency following a bone marrow transplantation.

Of particular significance for the first and other aspects of the invention is wherein the mammal, particularly a human, is asthmatic. As pointed out above the incidence of asthma is increasing and currently has a very significant health impact in western societies.

Inflammatory obstructive airways conditions typically include a quiescent period where the mammal is relatively unaffected, and an acute period where the inflammation is manifest and the airways become obstructed. The βc blocker in one form of this method is administered during the acute period. The method thus aims to reduce the severity of the attack. The time period of such attacks are not particularly extended, however as indicated above, a delayed phase of such inflammatory attacks can occur several hours after the early phase, and it may require two or more applications of the βc blocker over that time frame, and may be required to be continued for a day or longer to ensure that an AHR (airways hypersensitivity reaction) is not manifest.

The method of the first aspect of the invention may include the step of estimating the levels of IL-4 and/or IL-13 in the lung before administering the βc blocker, and the step of estimating the levels of IL-4 and/or IL-13 after administration of the βc blocker and calculating the reduction in IL-4 and/or IL 13. This then provides for a means for assessing the effectiveness of treatment, and if the treatment is on going with repeated or extended slow release administration of the βc blocker it provides for an assessment of whether there is a progressive reduction in reaction to, for example, an allergen.

Means of estimating the levels of IL-4 and or IL-13 may include immunological methods such as ELISA, or western blots, nucleic acid expression methods such as assessing the level of RNA for example, northern blotting. Alternatively indirect means may be used including estimating levels of the physiological effects of the condition.

One significant finding of the inventors is the extent to which blocking of βc signalling effects lung function in an inflammatory obstructive airways condition. It is thus found that early phase asthma attack is alleviated, and also very significantly that lung remodelling is inhibited to an extent that AHR (airways hyperresponsiveness) is not manifest on challenge with an allergen to which the lungs have been sensitised.

An extended cytokine imbalance in the lung leads to remodelling of the lung, which is associated with thickening of the airway walls by reason of fibrosis resulting from the deposition of collagen. This biosynthetic imbalance has been found to be disrupted by the blocking of βc signalling. The present invention thus provides for a means of inhibiting lung remodelling in a mammal that would otherwise result from an inflammatory obstructive lung condition. Reversing the imbalance is anticipated also to lead to a reversal of lung remodelling by altering the balance between collagen formation and breakdown. Over a period of time this should lead to a reduction in fibrosis of the lung, and thus sensitivity of the lung. It will be appreciated that this method has application to a range of conditions, symptoms of which result from fibrosis of the lung.

Severe cases of inflammatory obstructive airways conditions resulting from substantial lung remodelling are also refractory to treatment by conventional treatments including glucocorticosteroids and/or beta-agonists, anticholinergic agents, or other anti-inflammatory drugs the present method thus provides for a means of treating severe cases of asthma.

The second aspect the invention resides in a method of inhibition or reversing lung remodelling in a patient with an obstructive airways condition, the method including the step of administering an effective amount of a β_{c} blocker capable of blocking the binding of all three of IL-3, IL-5 and GM-CSF to the common β_{c} receptor, said β_{c} blocker being administered for a time sufficient to cause a reduction of lung remodelling.

It may be preferred to estimate the degree of lung remodelling, before administering the βc blocker and estimating the degree of lung remodelling after administering the β_{c} blocker and assessing the degree of reduction of the lung remodelling to give an indication of the extent to which treatment is effective.

The βc blockers contemplated and the methods of delivering the βc blocker are substantially as set out for the first aspect of the invention, except that administration may be preferred to be delivered during an extended time period.

The βc blocker is administered for a different temporal span in aiming to inhibit or reverse lung remodelling, as compared with treating the acute phase of an inflammatory obstructive lung condition. In asthma, even during the quiescent period Th2 cytokines are produced in the airways, which is somewhat contrary to typical inflammatory reactions because in the absence of recent antigen exposure a typical Th2 response should die down because normal regulatory functions eliminate effector CD4 T cells after activation. Quite why this is the case is not certain but it could be explained because antigen presentation may be prolonged owing to a small population of APC that can present antigen for up to eight weeks following inhalation exposure. It is therefore anticipated that in treatments of reduction of remodelling it is thus preferred to administer the βc blocker such that it is present in an effective amount in the pulmonary system for an extended period. This extended period is thus anticipated to be greater than for an acute attack, and may therefore be at least 1 or 2 days or preferably at least 3, 4, 5, 6, day and more preferably one or more weeks optionally 2, 3, 4, 5, 6, 7, 8, 9 or 10 weeks. Especially where the action of the βc blocker is preferentially in the pulmonary system, such that there is a reduced or minimal systemic effect the effective amount is present for at least 3 or more months, optionally for at least 4, 5, 6, 7, 8, 9, 10, 11 or 12 months.

The administration of the βc blocker may be by discrete repeated doses or by means of slow release application such as by provision of a dermal patch or other depot.

The second aspect of the invention may additionally include the step of estimating the time over which Th2 cytokines are elevated following the acute phase of the inflammatory airways condition, before administering the delivery of the βc blocker. It may also be desirable to monitor the level of one or more of the Th2 cytokines during the time over which the βc blocker is administered. The method may include thus administering the βc blocker at the onset of the attack and continuing the exposure beyond the attack until the level of one or more Th2 cytokines tapers off to a level that approaches or reaches basal levels in the individual. This period may vary as the treatment progresses.

In the alternative it may be desired to deliver the βc blocker as a slow release over an extended time frame of perhaps one or more months to perhaps about 12 months or more, and at the same time assessing the level of one or more Th2 cytokines that are elevated and the degree of lung remodelling and/or respiratory function. Desirably in this alternative form the βc blocker is preferentially delivered to the airways. Also preferably the method is characterized in that IL-4 and/or IL-13 levels are reduced from elevated levels associated with the inflammation.

Estimating the degree of lung remodelling may be achieved by 1) measuring the extent of the reaction by the mammal to the allergen by for example testing the extent of AHR. Alternatively it may be assessed by biopsy and histological examination of the biopsy sample. Arthroscopic examination of sample airways can be conducted. Respiratory function measurements provide a direct measure of lung blockage and thus lung remodelling is directly correlated with the extent of lung capacity. Lung capacity can be estimated as set out below. Respiratory function may be measured with and/or without exposure to the allergen concerned to ascertain the extent to which lung remodelling has occurred. Where measurement is simply without exposure to the allergen reference may be made to (age and/or gender adjusted) standard charts of lung capacity, alternatively a history of lung capacity may be compiled for the individual concerned and the improvement may be charted.

The second aspect of the invention will be understood in preferred forms to particularly relate to an inflammatory obstructive airways condition, and together with other aspects of this invention will be most applicable to one such condition namely asthma. Such condition may be one that is clinical where attacks are manifest so that the principal aim might be to ameliorate or prevent further onset of attacks. In the alternative such conditions may be sub-clinical condition where attacks are not manifest. This may be viewed as a preventative measure to reduce the prospects of attacks occurring, alternatively it may be used simply to enhance the respiratory capacity of the individual concerned as an enhancement of general health and well-being.

Respiratory function can be evaluated with a variety of static tests that comprise measuring a mammal's respiratory system function in the absence of a provoking agent. Examples of static tests include, for example, spirometry, plethysmographically, peak flows, symptom scores, physical signs (for example respiratory rate), wheezing, exercise tolerance, use of rescue medication (i.e., bronchodilators) and blood gases. Evaluating pulmonary function in static tests can be performed by measuring, for example, Total Lung Capacity (TLC), Thoracic Gas Volume (TgV), Functional Residual Capacity (FRC), Residual Volume (RV) and Specific Conductance (SGL) for lung volumes, Diffusing Capacity of the Lung for Carbon Monoxide (DLCO), arterial blood gases, including pH, P_{O2} and P_{CO2} for gas exchange. Both FEV₁ and FEV₁/FVC can be used to measure airflow limitation. If spirometry is used in humans, the FEV₁ of an individual can be compared to the FEV₁ of predicted values. Predicted FEV₁ values are available for standard normograms based on the mammal's age, sex, weight, height and race. A normal mammal typically has an FEV₁ at least about 80% of the predicted FEV₁ for the mammal. Airflow limitation results in a FEV₁ or FVC of less than 80% of predicted values. An alternative method to measure airflow limitation is based on the ratio of FEV₁ and FVC (FEV₁/FVC). Disease free individuals are defined as having a FEV₁/FVC ratio of at least about 80%. Airflow obstruction causes the ratio of FEV₁/FVC to fall to less than 80% of predicted values. Thus, a mammal having airflow limitation is defined by an FEV₁/FVC less than about 80%.

The effectiveness of a drug to protect a mammal having or susceptible to airflow limitation can be determined by measuring the percent improvement in FEV₁ and/or the FEV₁/FVC ratio before and after administration of the drug. In one embodiment, an effective amount of a βc blocker comprises an amount that is capable of reducing the airflow limitation of a mammal such that the FEV₁/FVC value of the mammal is at least about 80%. In another embodiment, an effective amount of a βc blocker comprises an amount that is capable of reducing the airflow limitation of a mammal such that the FEV₁/FVC value of the mammal is improved by at least about 5%, or at least about 100 cc or PGFRG 10 L/min. In another embodiment, an effective amount of a βc blocker comprises an amount that improves a mammal's FEV₁ by at least about 5%, and more preferably by between about 6% and about 100%, more preferably by between about 7% and about 100%, and even more preferably by between about 8% and about 100% (or about 200 ml) of the mammal's predicted FEV₁.

The third aspect the invention could be said to reside in a method of treatment or modulation of a severe inflammatory obstructive airway condition in a mammal, the condition being refractory to treatment with glucocorticosteroids, the method comprising the step of administering one or more times a βc blocker capable of blocking the binding of all three of IL-3, IL-5 and GM-CSF to the common βc.

The method of the third aspect additionally may include the administration of an additional active agent perhaps selected from the group comprising glucocorticosteroids, beta-agonists, anticholinergic agents or other therapeutic agents (including other immunotherapeutics) useful for alleviating the symptoms of the inflammatory obstructive airway condition in the mammal, in particular alveolar constriction.

The additional active agent may be administered together with the βc blocker together for example in aerosolized form in, for example, a “puffer” alternatively the βc blocker may be administered separately in any one of the forms set out for the second aspect above, perhaps conveniently in a slow release formulation, for example as a depot perhaps in the form of a skin patch depot or for mucosal release, or alternatively as an orally ingested slow release formulation. It is to be understood that the third aspect of the invention additionally includes variations set out with regards to other aspects of the invention.

The fourth aspect of the invention resides in a method of prescribing treatment for airway hyper-responsiveness and/or airflow limitation associated with a respiratory condition involving an inflammatory response in a mammal, comprising the steps of:
a. administering to the mammal a βc blocker capable of blocking the binding of all three of IL-3, IL-5 and GM-CSF to the common βc receptor,
b. measuring a change in respiratory function in response to an allergen in said mammal to determine if said βc blocker modulates airway hyperresponsivenss; and
c. prescribing a treatment comprising administering the βc blocker to the mammal in a dose effective to reduce inflammation based upon said changes in respiratory function.

A change in respiratory function includes measuring respiratory function before and after administration of a βc blocker. In accordance with the present invention, the mammal receiving the βc blocker is known to have a respiratory disease involving inflammation. Measuring a change in respiratory function in response to a provoking agent can be done using a variety of known techniques. In particular, a change in respiratory function can be measured by determining the FEV₁, FEV₁/FVC, PC₂₀methacholine FEV₁, post-enhanced pause (Penh), conductance, dynamic compliance, lung resistance (R_{L}), airway pressure time index (APTI), and/or peak flow for the recipient of the provoking agent. Other methods to measure a change in respiratory function include, for example, airway resistance, dynamic compliance, lung volumes, peak flows, symptom scores, physical signs (i.e., respiratory rate), wheezing, exercise tolerance, use of rescue medication (i.e., bronchodilators) and blood gases. A suitable pharmacological therapy effective to reduce inflammation in a mammal can be evaluated by determining if and to what extent the administration of a βc blocker has an effect on the respiratory function of the mammal. If a change in respiratory function results from the administration of a βc blocker, then that mammal can be treated with the βc blocker. Depending upon the extent of change in respiratory function, additional compounds can be administered to the mammal to enhance the treatment of the mammal.

A further aspect of the invention resides in a method for monitoring the success of a treatment in a mammal, for airway hyperresponsiveness and/or airflow limitation associated with a respiratory condition involving an inflammatory response, said method comprising:
a. administering an effective amount of a βc blocker to a mammal that has been treated for a respiratory disease involving an inflammatory response;
b. measuring a change in lung function in said mammal in response to a provoking agent; and
c. monitoring the success of said treatment by comparing said change in lung function with previous measurements of lung function in said mammal, or with a set standard.

This further aspect therefore assesses whether there are further gains to be had after treatment with either a conventional treatment, for example glucocorticosteroids or other treatments. Alternatively the assessment might be made after treatment with a method comprising the administration of a βc blocker either as the sole pharmacological agent or in combination with another pharmacological agent.

A consequence of the reduction in degree of IL-4 and IL-13 elevation following allergen challenge is that administration of a βc blocker also leads to a significant shift from Th2 immune reaction to a Th1 immune reaction. The data presented in the examples shows several indicators that blocking of βc signalling mediates immune deviation from a pathological Th2-dominated response towards a protective immune response in peripheral lymphoid tissues and in the lungs. Thus blocking of βc signalling dramatically decreases the effects of asthma associated allergies, including airway inflammation, eosinophilia and mucus production, significantly reduces antigen-specific IgE and IL-4 production, and increases IFN-γ levels. This finding has practical application in treatment prevention or modulation of inflammatory airways blockage conditions but it also has application for other allergic reactions.

Accordingly the fifth aspect of the invention resides in a method of biasing an immune response away from a Th2 immune response by administering a βc blocker capable of blocking the binding of all three of IL-3, IL-5 and GM-CSF to their common receptor to thereby change the levels of one or more markers indicative of a Th2 response.

The method preferably includes the step of measuring the one of more indicators of a Th2 response before administering the βc blocker, and the step of measuring the one or more markers indicative of the Th2 response after administering the βc blocker, and the step of comparing the two to calculate the change in levels of the one or more markers.

The one or more Th2 markers might be selected from the group consisting of IL-4, IL-5, IL-9, IL-10, and IL-13.

Most preferably levels of IL-4 and IL-13 are reduced.

A specific form of the fifth aspect relates to a respiratory condition, and the Th2 markers measured are present or derived from the pulmonary system, thus for example they may be present in the pulmonary system or have drained/migrated therefrom. Thus for example they may be assessed as being present in the PBLN (peribronchial lymph node).

The fifth aspect of the invention preferably includes the step of estimating the degree of proliferation of T-helper cells of the Th2 type in the host.

The fifth aspect is applicable to a range of conditions, particularly conditions involving an inflammatory or allergic condition, and in one form may be one of the group consisting of asthma, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumotitis, occupational asthma, sarcoid, reactive airway disease syndrome, interstitial lung disease, hyper-eosinophilic syndrome, rhinitis, sinusitis, or parasitic lung disease. The present invention is particularly applicable to asthma, emphysema, chronic bronchitis, and chronic bronchiolitis. Obstructive airways condition may include these and subclinical manifestations of these.

This may more particularly be applicable as a preventative measure so that where an individual is placed in an environment with a high risk of developing an allergy, perhaps during a season where developing “hayfever” to known airborne allergens is a high risk, or alternatively in certain occupations.

Alternatively this may be used as a treatment to reverse an already existing allergy, whereby the βc blocker is administered during onset of the allergy or in a high risk environment where there is a high risk of onset of the allergy. Thus with a food allergy the βc blocker may be administered to coincide with ingestion of the food or at least such that the binding to βc of all three of GM-CSF, IL-3 and IL-5 is blocked at the time of food ingestion.

It is to be understood that the fifth aspect of the invention additionally includes variations set out with regards to other aspects of the invention.

The sixth aspect of the invention resides in a method of converting an established antigen-specific allergic response characterized by the production of Th2-type cytokines to a Th1-type response, the method comprising:
administering an effective dose of allergen in conjunction with a βc blocker for a period of time sufficient to convert the allergen-specific allergic response to a Th1-type response,
the βc blocker capable of blocking the binding of all three of IL-3, IL-5 and GM-CSF to their common receptor to thereby change levels of one or more markers indicative of a Th2-type response.

Allergens are immunogenic compounds that cause Th2-type T cell responses and IgE B cell responses in susceptible individuals. Allergens of interest according to the present invention include antigens found in foods such as fruits (e.g., melons, strawberries, pineapple and other tropical fruits), peanuts, peanut oil, other nuts, milk proteins, egg whites, shellfish, tomatoes, etc.; airborne antigens such as grass pollens, animal danders, house mite feces, etc.; drug antigens such as penicillins and related antibiotics, sulfa drugs, barbituates, anticonvulsants, insulin preparations, local anaesthetics, and iodine; insect venoms and agents responsible for allergic dermatitis caused by blood sucking arthropods such as Diptera, including mosquitos, flies particularly black flies, deer flies and biting midges, ticks, fleas; and latex. The specific allergen may be any type of chemical compound such as, for example, a polysaccharide, a fatty acid moiety, a protein, or the like. Antigen preparations may be prepared by any available technique including, for example, isolation from natural sources, in vivo or in vitro expression of recombinant DNA molecules, chemical synthesis, or other technique known in the art.

The most common anaphylactic allergens include food allergens (especially peanut allergens), insect venoms, drug allergens, and latex.

It is appropriate to deliver the allergens via their normal route, to get to the organ that is most affected. Thus food allergens are preferably delivered orally, skin allergies may be delivered dermally (perhaps as creams) and allergies of the airways are to be delivered via the lung. These are the most likely routes of delivery, however other routes may also be applicable, particularly if delivered to a mucosal surface.

One particular focus of this invention are respiratory conditions because it is found that there is preferential reduction in IL-4 and IL-13 in the lung, and the present invention is particularly relevant to inflammatory obstructive airways conditions more particularly allergic obstructive airways conditions.

The amount of allergen preparation to be administered in preventive immunotherapy protocols may be empirically determined, and will depend, among other things, on the size of the recipient. Usually, at least about 100 ng of allergen will be required per kg of body weight, but more than 1 mg/allergen/kg body weight will usually not be desirable. Administration schedules may vary with individual patients, and may include periodic increases to the amount of allergen administered, optionally by as much as about ten to one hundred fold.

One specific form of the sixth aspect relates to the treatment of asthma and thus the invention might be said to reside in a method of treating asthma associated allergies, the method comprising:
administering to a patient an effective dose of an asthma associated allergen in conjunction with a βc blocker, the βc blocker capable of blocking the binding of all three of IL-3, IL-5 and GM-CSF to their common receptor;
wherein the effects of the asthma associated allergies are decreased.

The asthma associated allergen may be selected from one or a combination of more than one of the group consisting of house dust mite, cat and cockroach allergens, pollen and plant allergens.

It is to be understood that the sixth aspect of the invention additionally includes variations set out with regards to other aspects of the invention.

Various features of the invention have been particularly shown and described in connection with the exemplified embodiments of the invention, however, it must be understood that these particular arrangements merely illustrate and that the invention is not limited thereto and can include various modifications falling within the spirit and scope of the invention.

### EXAMPLES

The present example uses mice with a targeted disruption in both the βc and β_{IL-3} genes to characterise the response to allergen sensitisation and challenge in mice entirely deficient for this receptor. We demonstrate for the first time that prevention of βc signalling significantly precludes the hallmark features of allergic airways disease, reducing airways hyperresponsiveness and pulmonary eosinophilia down to baseline levels. Further, the early phase serum IgE response and mucus hypersecretion are attenuated, and the ability of CD4+ T cells to produce Th2 cytokines in the pulmonary compartment is reduced, in the absence of effects on systemic immunity. Thus, targeting βc is not only able to influence eosinophil function but also impact on the intrinsic signals that govern Th2 effector cell function and consequently bronchoconstriction.

### Materials and Methods

### Mice

Mice deficient for the IL-3/IL-5/GM-CSF β common receptor subunit and the IL-3β subunit (βc/β_{IL-3} double knockout) were supplied by Prof. Angel Lopez at the Institute of Medical and Veterinary Science (IMVS), Adelaide, Australia. The absence of the β_{IL-3} receptor eliminates residual IL-3 signalling in the absence of βc, thus more accurately represented the human therapeutic setting, where the β_{IL-3} receptor is absent and inactivation of βc would be eradicate all signalling through all three cytokines (IL-3, IL-5 and GM-CSF). These mice are referred to as βc−/− throughout this document. Wild-type (WT) BALB/c mice were obtained from the University of Newcastle, Callaghan, Australia. All mice were housed under specific pathogen free conditions, and all procedures were subject to approval by the University of Newcastle Animal Care and Ethics Committee (ACEC).

### Induction of Allergic Airways Disease by OVA Sensitisation

6-8-wk-old mice were sensitized by intraperitoneal injection of 50 μg of ovalbumin (OVA) with 1 mg alhydrogel (CSL Ltd.) in 0.9% sterile saline (“allergic” group). Nonallergic mice received 1 mg alhydrogel in 0.9% saline. On days 12, 13, 14, and 15, all groups of mice were aeroallergen challenged by intranasal instillation of 10 μg OVA in 0.9% saline under light isofluorane anaesthesia. In most experiments, AHR was measured 24 h after the final challenge. Mice were then sacrificed by sodium pentobarbital overdose and T cell and humoral responses, cellular profiles, inflammation and morphological changes to the airways characterized. In experiments addressing the temporal infiltration of eosinophilia in allergic airways disease, mice were sacrificed at 24 h (day 1), day 2, day 3, day 7 and day 14 after final antigen challenge.

### Measurement of Airways Hyperreactivity (AHR)

Airway hyperreactivity to inhaled β-methacholine representative of the large (Transpulmonary resistance, R_{L}) and small (dynamic compliance, C_{dyn}) airways was determined. Animals were anaesthetized by intraperitoneal injection of ketamine-xylazine and tracheostomized with insertion of a polyethylene cannula (i.d. 0.813 mm). The tracheal tube was connected to a ventilation port within the plethysmograph chamber, and this port was connected to a rodent ventilator (HSE Minivent Type 845, Hugo Sachs Elektronik, Harvard, Germany). Mice were mechanically ventilated at a rate of 140 breaths per minute with a stroke volume of 180 μl. Volume changes due to thoracic expansion with ventilation were measured by a transducer connected to the plethysmograph flow chamber. A pressure transducer measured alterations in tracheal pressure as a function of airway calibre. Once stabilized, mice were challenged with saline, followed by increasing concentrations of β-methacholine (6.25, 12.5, 25 and 50 mg/ml). Aerosols were generated with an ultrasonic nebuliser (Buxco, Aeroneb Laboratory Nebulizer) and delivered to the inspiratory line. Each aerosol was delivered for a period of 5 minutes, during which pressure and flow data were continuously recorded, and specialist software (BioSystemXA, Buxco Electronics, Inc.) was used to calculate pulmonary resistance and compliance. Peak values were taken as the maximum response to the concentration of methacholine being tested, and were expressed as the percentage change over the saline control.

### Analysis of Inflammatory Cells in Blood and Bronchoalveolar Lavage Fluid (BALF)

Immediately after sacrifice, blood was collected by cardiac puncture and a small aliquot used to prepare a blood smear. Slides were stained with May-Grunwald Giemsa and differential leukocyte counts performed based on morphological criteria (minimum 200 cells counted per slide). The remaining blood was centrifuged (10,000 g, 10 min) and serum collected and stored at −70° C. until analysis. Bronchoalveolar lavage fluid (BALF) was obtained by cannulating the trachea and gently flushing the airways with two 1 ml volumes of Hanks Buffered Salt Solution (HBSS). Recovered cells were pelleted by centrifugation, resuspended in erythrocyte lysis buffer for 5 min, then washed and counted to determine the total number of cells recovered. May-Grunwald Giemsa-stained cytospins were prepared and differential leukocyte counts performed based on morphological criteria (minimum 200 cells counted per slide).

### Characterization of Eosinophils and Mucus-Staining Cells in Lung Tissue

Lung tissue representing the central (bronchi-bronchiole) and peripheral (alveoli) airways were fixed in 10% phosphate-buffered formalin, sectioned, and stained with Carbol's chromotrope-hematoxylin for identification of eosinophils or alcian blue/periodic acid-Schiff for enumeration of mucin-secreting cells. The mean number of eosinophils or mucus secreting cells (MSC) per high-powered field (HPF; ×100 magnification) within 100 μm of the basement membrane was determined following assessment of a minimum of 10 HPF.

### Measurement of Cell Proliferation and Cytokine Production in Peribronchial Lymph Nodes (PBLNs) and Spleens

PBLNs and spleens were excised and filtered through 70 μm nylon mesh. The filtrate was then centrifuged at 500 g for 5 min at 4° C. and the cell pellet resuspended in erythrocyte lysis buffer and the centrifugation repeated. The resulting cell preparation was cultured at 37° C./5% CO₂ in 96-well plates at 1×10⁶ cells/well in animal cell culture medium (ACCM; 0.1 mM sodium pyruvate, 2 mM L-glutamine, 20 mM HEPES, 100 U/ml penicillin/streptomycin, 50 μM 2-mercaptoethanol and 10% fetal bovine serum in RPMI 1640) in the presence of 200 μg/ml OVA (200 μl/well final volume). Unstimulated wells contained cells and culture media only, in the absence of antigen stimulation. For measurement of cell proliferation, cultures were incubated for 72 h after which proliferation was determined with the Cell-Titre 96 reagent (Promega) following the manufacturer's instructions. Antigen-specific proliferation was calculated as the percentage proliferation in antigen-treated wells compared to unstimulated wells from the same cell preparation.

For analysis of cytokine production, cultures were incubated for 6 d and cell-free culture supernatants collected and stored in aliquots at −70° C. until analysis. IL-5, IL-4, IFN-γ (all from BD Pharmingen) and IL-13 (R&D Systems) concentrations were determined in culture supernatants by ELISA according to the supplier's recommendations.

### Determination of Antigen-Specific Serum Immunoglobulins by ELISA

OVA-specific IgGᵢ and IgG₂ₐ levels were semi-quantified by ELISA using reagents from BD Pharmingen. Briefly, plates were coated overnight at 4° C. with either OVA (2 μg/well in NaHCO₃ buffer, pH 9.6) for sample wells or unlabelled anti-IgG of corresponding isotype for standard wells. Plates were blocked with 3% BSA in PBS for 1 h at 37° C. All subsequent incubations were performed in 1% BSA/PBS diluent at 37° C. After incubation with serum samples or standards (mouse IgGᵢ or IgG₂ₐ) for 1.5 h, immunoglobulins were detected with streptavidin-horseradish peroxidase-(HRP-) conjugated anti-IgGᵢ or anti-IgG₂ₐ for 1 h. Plates were developed with tetramethyl-benzidine substrate solution (Sigma), the reaction stopped with 0.3 M H₂SO₄ and absorbances determined at 450 nm using a BioRad 680 Microplate reader.

Relative levels of OVA-specific IgE were determined by ELISA. Plates were coated overnight at 4° C. with unlabelled anti-IgE (BD Pharmingen) and blocked with 10% FCS in PBS for 1 h at 37° C. All subsequent incubations were performed in 10% FCS/PBS diluent at 37° C. After incubation with serum samples for 2 h, OVA-specific IgE was detected using OVA labelled with biotin (Pierce Biosciences; labelling performed according to manufacturers instructions) followed by streptavidin-HRP (Biosource) for 1 h each. Plates were developed as described above and absorbances at 450 nm used to calculate ELISA units relative to standardized positive and negative control serum.

### Lymphocyte and Dendritic Cell Profiling by Flow Cytometry

The phenotype of lymphocytes and dendritic cells in PBLN and lung samples were determined by flow cytometry using antibodies from BD Pharmingen. PBLN cell suspensions were prepared as described above. Lungs were excised and homogenates prepared by mechanical maceration followed by incubation in 1 mg/ml collagenase for 30 min at 37° C. Tissue was filtered through nylon mesh (70 μm) and the filtrate centrifuged at 500 g for 5 min at 4° C., cell pellet resuspended in erythrocyte lysis buffer then the centrifugation repeated. PBLN and lung cells were resuspended in staining buffer (1% BSA in PBS) and plated at 1×10⁶ cells/well into 96-well plates. Following 20 min incubation with Fc blocking antibody, cells were stained with fluorochrome-conjugated antibodies for analysis of lymphocytes (CD3, CD4, CD8, B220 and CD69) and dendritic cells (CD11c, CD11b, GR-1, PDCA-1, MHC II, CD80 and CD86).

Labelled cells were fixed in 1% paraformaldehyde and analysed using a BD FACSCanto™ flow cytometer.

In Vitro Th2 Polarization of Naïve CD4+T Cells

Splenocytes were prepared from naïve βc−/− and WT mice as described above and CD4+ T cells isolated by positive selection using magnetic beads (BD Pharmingen). Purified cells were cultured at 37° C./5% CO₂ in ACCM at 2×10⁵ cells/well in the presence of anti-CD3 (50 ng/ml; clone 2C11), anti-CD28 (1 μg/ml, clone 37.51), recombinant murine IL-4 (20 ng/ml), and anti-IFN-γ (40 μg/ml; clone R46A2) for 4 d to generate Th2-polarised populations. Cells were then washed and restimulated in the presence of anti-CD3 (50 ng/ml) and anti-CD28 (1 μg/ml) in 96-well plates (2×10⁵ cells/well, 200 μl/well final volume) for 6 d. Cell-free culture supernatants were collected and stored in aliquots at −70° C. until analysis of cytokine levels by ELISA.

### Statistical Analysis

The significance of differences between experimental groups was analysed using Student's unpaired t test. Values were reported as the mean±SEM. Differences in means were considered significant if p<0.05.

### Results

### Example 1

### Attenuation of Signalling Through the IL-3/IL-5/GM-CSF βc Receptor Suppresses Aeroallergen-Induced Eosinophilia

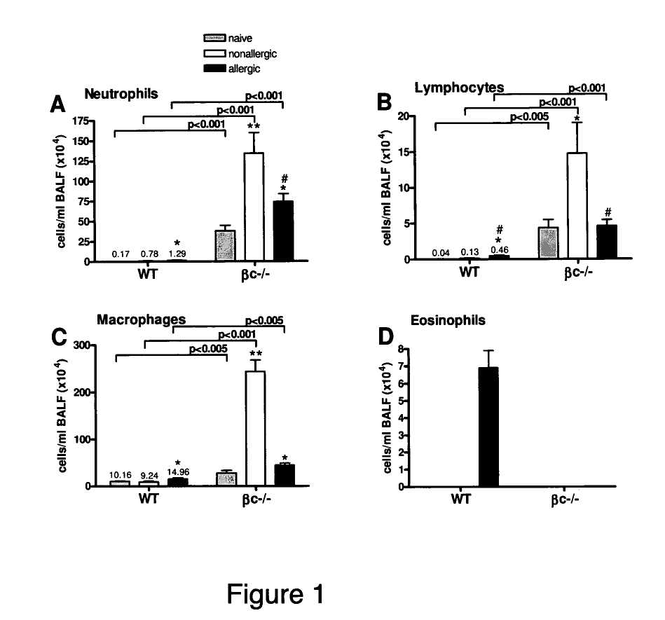

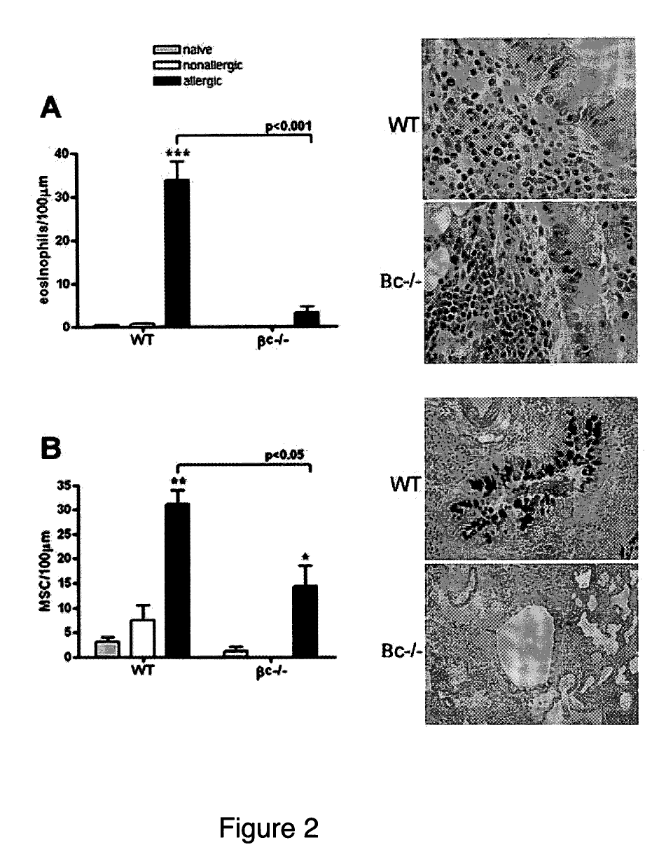

To determine the impact of βc deficiency on eosinophil expansion and migration to the airway in response to antigen inhalation, numbers of this leukocyte were measured in the blood, pulmonary tissue and BALF (Bronchoalveolar Lavage Fluid) fluid of allergic mice deficient in this molecule. Eosinophil expansion was observed in the blood of allergic WT mice (9.4%±2.0) compared to their nonallergic counterparts (1.7%±0.5). Further, eosinophils migrated to the pulmonary compartment in WT mice, accumulating in the peribronchial tissue (FIG. 2, A) and airway lumen (FIG. 1, D). By contrast, eosinophilic infiltrates in the blood (0.3%±0.1) and lung tissue (FIG. 2, A) of βc−/− mice were reduced to levels analogous to that observed in WT nonallergic mice. Notably, this granulocyte was entirely absent from the BALF (FIG. 1, D).

Differential leukocyte analysis of BALF revealed extensive infiltration of neutrophils, lymphocytes and macrophages in the βc−/− airway, both at baseline and in the allergy model compared to the WT (FIG. 1, A-C). Previous phenotypic analysis of mice with a null mutation for βc revealed a lung pathology resembling the human disease pulmonary alveolar proteinosis (PAP), characterised histologically by the presence of foamy macrophages and necrotic cellular debris in the airways and thought to be mediated by progressive accumulation of surfactant protein due to ineffective alveolar macrophage function (46, 47, 51). This defect is shared by mice lacking the GM-CSF ligand and thus appears to be a feature of total elimination of signalling by this cytokine (52, 53). Alveolar proteinosis is likely to contribute to the presence of inflammatory infiltrates in naïve βc−/− mice in our study. It should be noted that in βc−/− mice, an increase in neutrophils and macrophages between naïve and allergic mice reminiscent of that seen in WT mice is observed (FIG. 1, A & C). Nonallergic βc−/− mice, challenged with antigen in the absence of sensitisation, show a significant inflammatory infiltrate (FIG. 1, A-C). This may be a feature of non-specific activation of the inflammatory response in response to dosing PAP lungs with antigen.

### Example 2

### Absence of Airways Hyperreactivity and reduced Pulmonary Mucus Secretion Following Antigen Provocation in βc−/− Mice

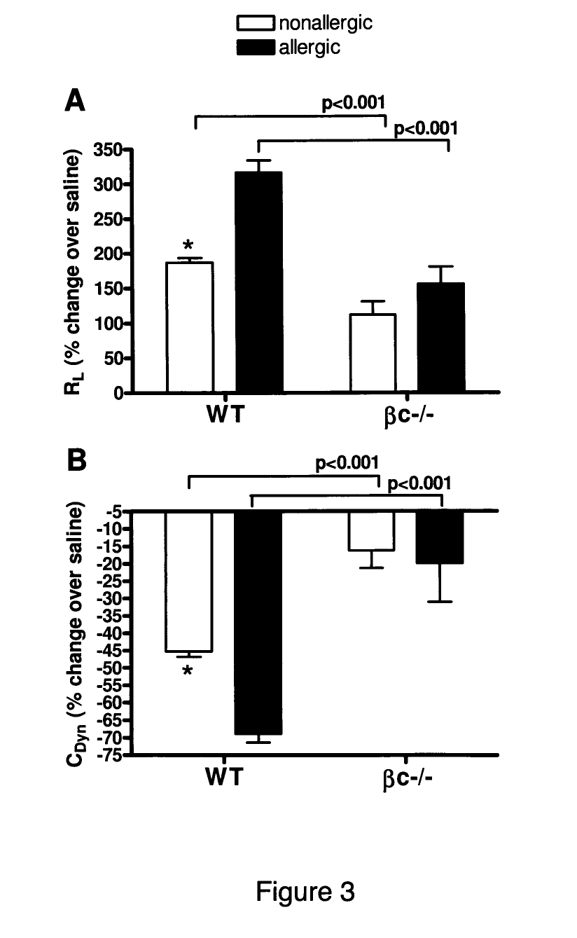

Antigen inhalation induced a marked airways hyperreactivity (AHR) to β-methacholine in allergic WT mice, measured by an increase in transpulmonary resistance (R_{L}) and a decrease in dynamic compliance (C_{dyn}) of the airways (FIG. 3). The dose indicative of the maximal response to β-methacholine is shown, which is also representative of the entire dose-response curve. By contrast, βc−/− mice fail to develop AHR following allergen sensitisation and airway challenge (FIG. 3). Further, although significantly abrogated, mucus hypersecretion was still a notable feature in the lung of allergic βc−/− mice (FIG. 2, B). However, although the pattern of expression of mucus secreting cells in WT lungs commonly presented as a high frequency of cells within a single, highly inflamed airway causing visible obstruction of the lumen, histological examination of βc−/− mice revealed that the mucus secreting cells (MSC) present were disseminated throughout the tissue, and the threshold of MSC in any one individual airway did not appear to cause significant obstruction (FIG. 2, B). This observation is supported by the absence of airway occlusion noted during AHR measurements in allergic βc−/− mice (FIG. 3).

### Example 3

Pulmonary Th2 Cytokine Release is reduced in the Absence of βc

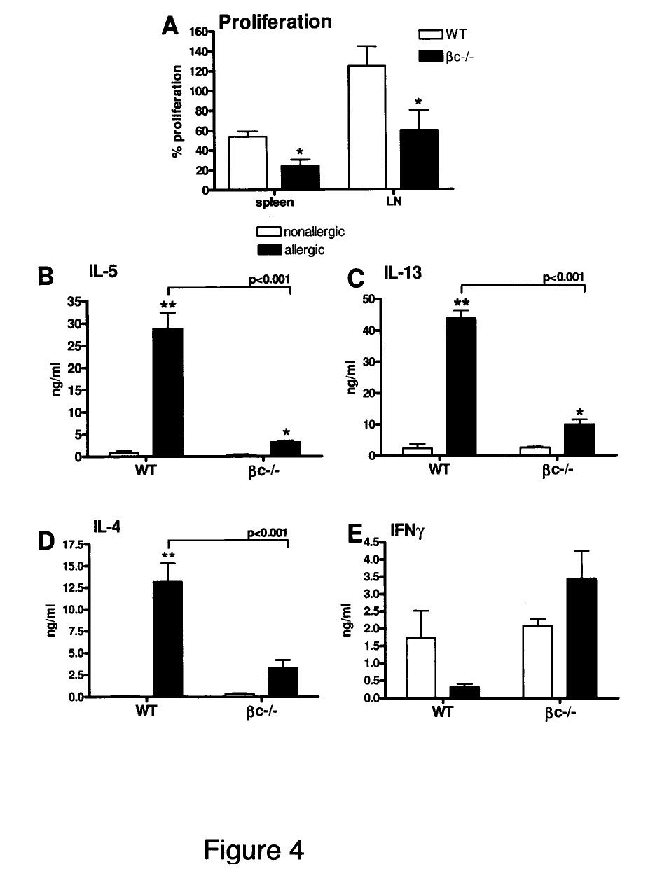

It is well established that signals elicited by CD4+T2 cells perform an obligatory role in the induction of allergic airways disease. For this reason we investigated the impact of 13c deficiency on proliferation and the liberation of hallmark Th2 cytokines from both local (PBLN) and systemic (spleen) sites from allergic mice following antigen restimulation in vitro. The ability of cells from both the spleen and PBLN to proliferate in response to antigen was diminished in βc−/− mice relative to their wild-type counterparts (FIG. 4, A). Nonetheless proliferation levels in βc−/− mice remain higher than that of the nonallergic WT, suggesting that these cells retain an inherent proliferative capacity in the absence of IL-3/IL-5/GM-CSF signalling (data not shown). Importantly, βc inactivation is accompanied by a striking reduction in the antigen-specific production of IL-5, IL-13 and IL-4 in PBLN cultures (FIG. 4, B-D). This effect appeared to be a localised response in the pulmonary compartment, with no global reduction in Th2 responses observed in splenocyte populations (data not shown). Levels of IFNγ, a key determinant in type 1 CD4+ T cell responses, were also quantified (FIG. 4, E). Although a trend towards enhanced production of this cytokine is evident in βc−/− mice, this is not significant. It is important to note that the absolute levels recorded are considerably lower than those mounted in response to challenge with Th1 stimuli (IFN-γ ˜250 ng following viral infection, personal observation), and thus these results could most accurately be described as a suppression of Th2 cytokine production rather than bias towards Th1 immune responses.

### Example 4

### Inactivation of βc Signalling Suppresses Antigen-specific IgE and IgG₂ₐ but Augments IgG₁ Production

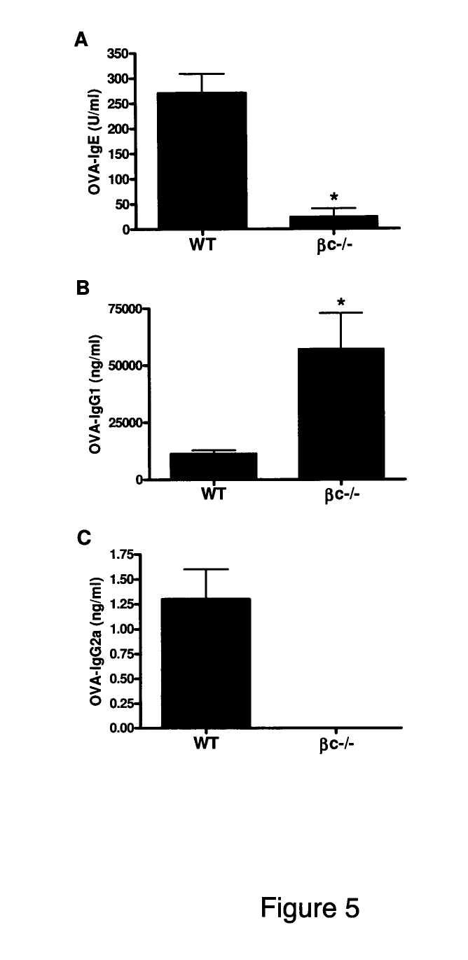

It is well documented that elevated serum antigen-specific IgE and IgG₁ production is linked to Th2 polarisation and the allergic phenotype, in the context of both in vivo experimental models and the clinical setting. The increase in antigen-specific IgE observed in the allergic airway is most likely orchestrated by a βc-dependent mechanism, as mice null for this receptor show significant attenuation in levels of this immunoglobulin in the serum (FIG. 5, A). βc−/− mice generated significantly higher levels of OVA-IgG₁ and reduced OVA-IgG₂ₐ in relation to the WT allergic mouse (FIGS. 5, B-C). This is contrary to dampening of Th2 responses implied by the PBLN cytokine profile (FIGS. 4, B-D) and may represent a previously undescribed regulatory effect of IL-3/IL-5/GM-CSF signalling on B cell function that warrants further investigation.

### Example 5

### Lack of Infiltration of Eosinophils is Not Delayed in βc−/− Mice

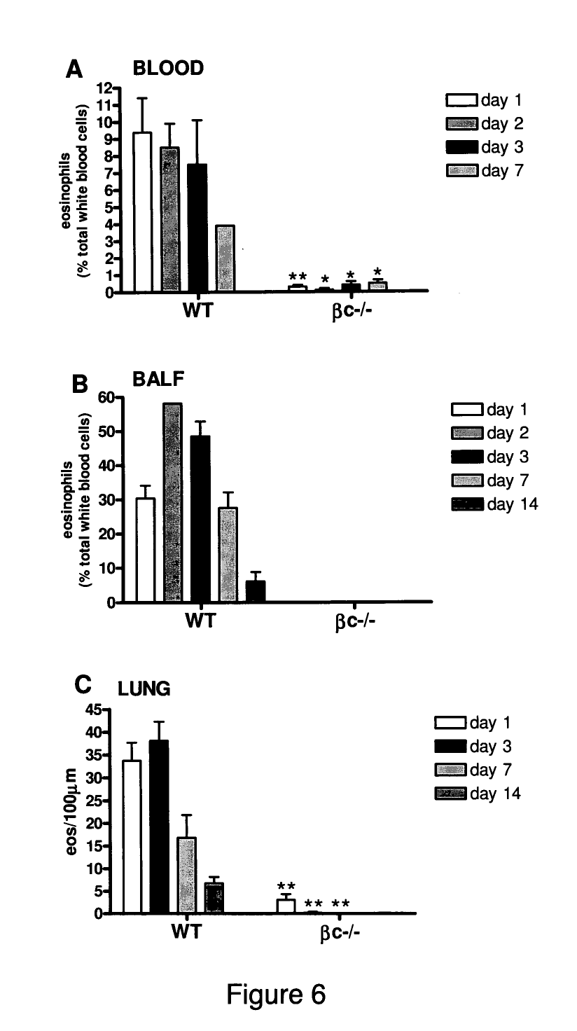

Previous examination of the immune response to parasite challenge revealed that in βc null mice, eosinophil expansion is delayed and significantly attenuated compared to WT controls (47). This suggests that βc−/− mice retain the ability to recruit eosinophils in response to parasites via residual signalling through the β_{IL-3} receptor, although to a lesser extent and by slower kinetics, and raises questions regarding the temporal pattern of eosinophilic infiltration in the allergic airways disease model. Although the appearance of eosinophils in the lung is clearly reduced 24 h after final aeroallergen challenge (FIGS. 1 and 2), further exploration beyond this time point was warranted. By contrast to the parasite infection model, eosinophil responses to pulmonary allergen provocation in the peripheral blood, airway lumen and lung tissue remained abrogated as far as day 14 after final challenge, at which time WT responses are virtually resolved (FIG. 6, A-C). The mild, delayed eosinophil response in the parasite infection model may be due to residual IL-3 signalling through the β_{IL-3} receptor. The elimination of both βc and β_{IL-3} signalling in our model by using double knockout mice may explain the absence of an eosinophil response in our allergy model, even two weeks after antigen challenge, and is a more accurate reflection of the signalling mechanisms in the human.

### Example 6

### Limitations in the Intrinsic Ability of Naïve βc−/− CD4+ T cells to Polarise to a Type 2 Phenotype

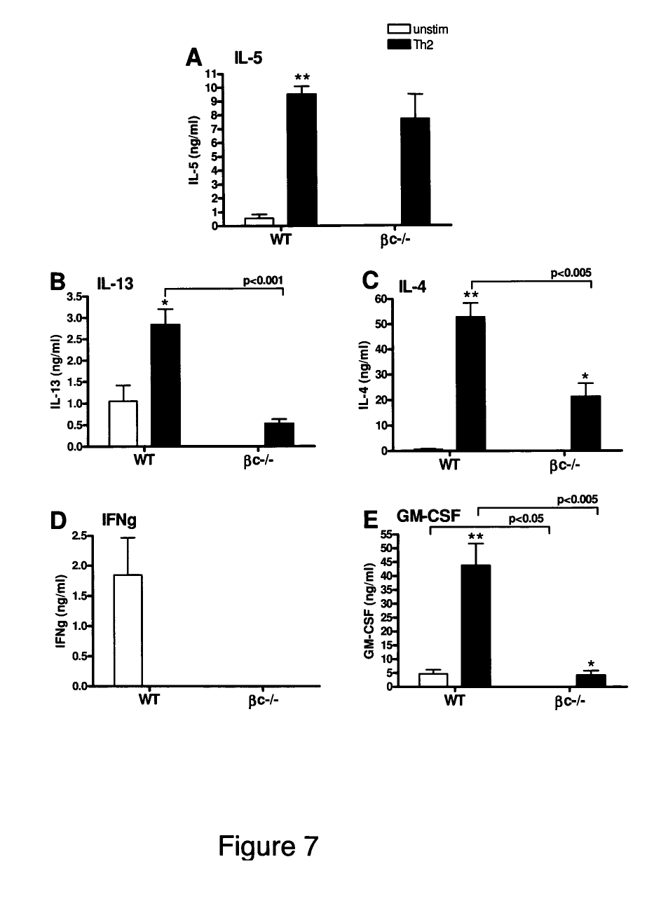

The reduced pulmonary Th2 cytokine production observed in allergic βc mice provokes speculation on the mechanism governing the suppression of T cell responses in vivo. Experiments were performed to address whether these data could be explained by a decline of the intrinsic ability of T cells from βc−/− mice to respond to antigen and liberate cytokines, or alternatively by defects specific to the pulmonary compartment of these mice. Naïve CD4+ T cells were isolated from the spleens of WT and βc−/− mice and cultured under biased conditions designed to promote Th2 cell differentiation in vitro. After 6 days of culture, βc−/− CD4+ T cells were limited in their ability to produce IL-13, IL-4 and GM-CSF compared to WT controls (FIG. 7, B-E). No significant difference in IL-5 secretion was observed (FIG. 7, A) This supports the premise that intrinsic defects in cytokine signalling in the absence of the βc receptor contribute to the inhibition of airway pathology observed in the in vivo model.

### Example 7

### Activated Lymphocytes Fail to Migrate to the Lung in the Absence of βc Signalling

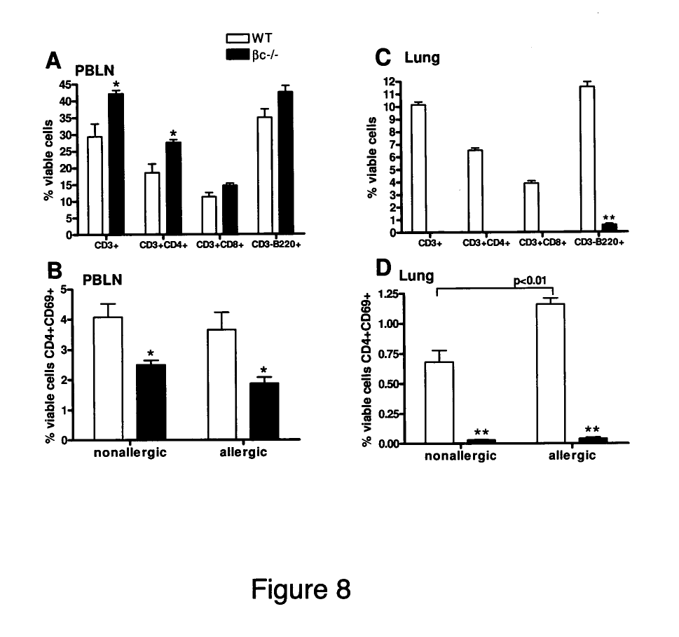

Flow cytometry was employed to determine the profile of lymphocytes in the PBLN and lung of allergic mice and thus determine the contribution of T cell migration and activation to the inhibition of allergic disease in βc−/− mice. PBLN and lung homogenates were prepared from allergic mice 24 h after the final antigen challenge and stained for total T cell numbers (CD3+), CD4+ T cells, CD8+ T cells and B lymphocytes (CD3-B220+). Interestingly, despite demonstrating low levels of cytokine production, βc−/− PBLN cell populations comprise significantly more T cells than their WT equivalents (FIG. 8, A). This increase can primarily be accounted for by CD4 positive lymphocytes, although a mild but insignificant increase in CD8+ T cell and B lymphocyte numbers in βc−/− allergic lymph nodes was noted. Despite a rise in overall cell number, the population of activated (CD4+CD69+) lymphocytes recovered from the PBLN of βc−/− mice was significantly reduced in both nonallergic and allergic mice (FIG. 8, B). The absence of an increase in CD69 expression between nonallergic and allergic PBLN cell preparations may relate to the specific time point at which sampling occurred. After 4 days of antigen challenge, expression of this early activation marker may have been downregulated in the lymph node, which is the site of initial antigen presentation and costimulation by airway dendritic cells. Alternatively the pool of activated CD4+ cells in the allergic lymph node may have migrated to the lung, as significant increases in CD4+ CD69+ cell numbers between nonallergic and allergic mice are observed in lung homogenates (FIG. 8, D).

By contrast to the PBLN profile, both T and B lymphocytes are virtually absent from the βc−/− lung (FIG. 8, C). Of particular note is the dearth of activated effector CD4+ lymphocytes in the lung, which is significant as these cells contribute significantly to allergic inflammation. These data suggest that defects in lymphocyte migration and activation in the absence of βc receptor signalling may give rise to a microenvironment that is protective against the development of inflammatory airways disease.

### Example 8 βc Expression Modulates the Number of and Expression of Costimulatory Molecules by Dendritic Cells in the PBLN and Lung

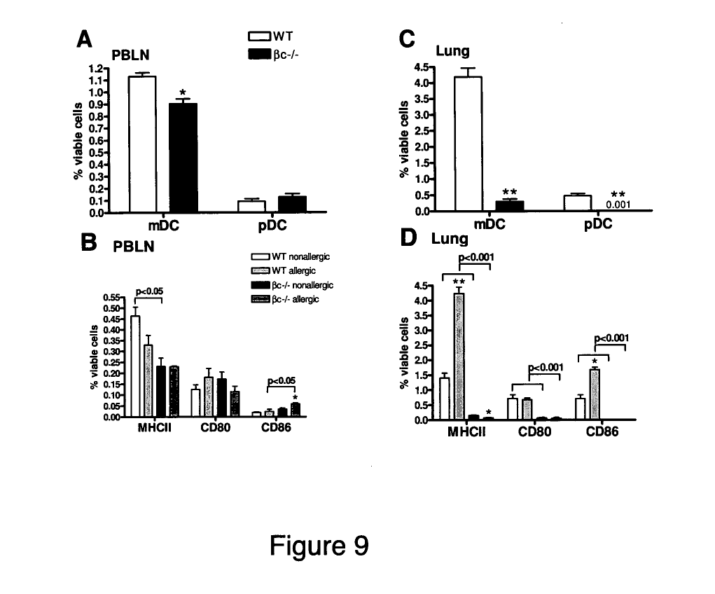

Synergistic antigen presentation and costimulation of CD4+ lymphocytes by myeloid dendritic cells (mDCs) in the airway has been implicated in the activation and migration of lymphocytes to the lung during the allergic response to inhaled antigen. Our data describing the abrogation of CD4+ T cell migration and activation in βc−/− mice raises important questions about the functionality of the DC pool in the absence of IL-3/IL-5/GM-CSF signalling through the βc receptor. We used flow cytometry to examine the incidence of mDCs (CD11c+CD11b+) and pDCs (CD11c+CD11b-GR-1-PDCA1+) in the lungs and the draining lymph nodes, and the expression a costimulatory molecules (MHC II, CD80 and CD86) in allergic WT and βc−/− mice. Inactivation of the βc receptor in allergic mice diminishes mDC numbers in the PBLN relative to allergic WT controls (FIG. 9, A) to a level not significantly different from the numbers of resident mDCs observed in naïve WT mice (0.85%±0.03 of total viable cells). No effect of receptor inactivation on pDCs numbers in the PBLN was evident (FIG. 9, A). Furthermore a striking decrease in mDC in the lungs of βc−/− mice is apparent (FIG. 9, B), with cell numbers significantly lower than that observed in the naïve WT (2.28%±0.20) suggesting a decline in both endogenous and allergen-induced pulmonary dendritic cell populations. Interestingly, lung pDCs are also reduced in the absence of fβc (FIG. 9, B).

Although the relationship between allergic disease and increased surface expression of costimulatory molecules was not clear in the PBLN, lung mDCs showed significant increases in MHC II and CD86 expression in WT allergic mice (FIG. 9, C-D). On the contrary, functional mDCs are virtually absent from the lung in the absence of βc signalling (FIG. 9, D). Collectively, these data suggest defects in the expansion and maturation of dendritic cells in the lung microenvironment at baseline and in inflammatory conditions in βc−/− mice.

### Discussion

In examples 1 to 8 we provide evidence to support the premise that the IL-3/IL-5/GM-CSF common β receptor chain (βc) is a valid target for the attenuation of allergic lung inflammation, such that in the absence of this molecule there is a reduction in all of the hallmark pathophysiological features of the disease. Further, we show that the mechanisms underlying the attenuation of disease involve fundamental defects in effector Th2 cell function and recruitment of both T lymphocytes and dendritic cells to the pulmonary compartment following allergen provocation.

Mice with targeted disruption of the βc gene (βc−/−) have previously demonstrated reduced eosinophil development from bone marrow progenitors at baseline conditions and in response to parasite infection (46, 47). Our studies demonstrate for the first time that the absence of βc signalling prevents the development of peripheral blood, peribronchial tissue and airway lumen eosinophilia following antigen sensitisation and challenge in a model of allergic airway inflammation (FIGS. 1, 2A). The data thus confirms that the βc receptor represents a critical molecular switch for the regulation of eosinophil biology, both endogenously and in inflammatory conditions, and we show for the first time that no redundant pathways exist for expansion of this granulocyte from the bone marrow. It therefore appears that βc signalling is a critical component for eosinophil expansion in allergic inflammation in vivo.

Although eosinophils are reduced, levels of other inflammatory cells in the airway are elevated in βc−/− mice compared to their wild-type counterparts (FIG. 1). This can most likely be attributed to background inflammation resulting from the alveolar proteinosis that is a phenotypic feature of the knockout mouse (46, 47). However, the increases in neutrophils and macrophages in allergic βc−/− mice compared to naives resemble that seen in the wild-type (FIG. 1).

By contrast to previous studies targeting IL-5 in mice with a BALB/c background (25), the reduction in eosinophilia in βc−/− mice was accompanied by an abrogation in bronchial hyperresponsiveness. Airway sensitivity to β-methacholine in antigen challenge βc−/− mice was reduced to levels analogous to the non-allergic WT (FIG. 3). Further, mucus hypersecretion was also reduced, albeit not to baseline levels (FIG. 2B). Nonetheless the expression pattern of mucus-positive material did not suggest consequent airway obstruction, an observation which is further substantiated by the apparently normal lung function of these mice (FIG. 3).

Having established that βc−/− mice do not develop key pathophysiological features of asthma, it is of significant interest to elucidate the underlying immunological mechanisms. Expansion, cytokine secretion and pulmonary migration of CD4+ T helper-2 (Th2) effector cells is an accepted paradigm in allergic airways disorders, the importance of which has been demonstrated in clinical manifestations of the disease. Our data demonstrate that systemic and local peribronchial lymph node (PBLN) T cells retain the ability to proliferate in response to antigen in the absence of βc signalling, though at a reduced capacity in relation to the WT (FIG. 4, A). Nevertheless although the ability to respond to antigen remains intact, cells in the βc−/− PBLN possess a strikingly reduced capacity to produce the Th 2 cytokines IL-5, IL-13 and IL-4 (FIG. 4, B-D), although liberation of the type 1 cytokine IFN-γ remains unchanged (FIG. 4, E). βc inactivation has not only influenced IL-5, but has impacted on signalling through the other major cytokine pathways in asthma, the IL-4/IL-13 axis, such that targeting of a single molecule has the potential to reduce secretion of a broad range of Th2 cytokines known to be associated with the deleterious effects of the disease. Interestingly, this global decrease in Th2 cytokines is not mirrored in spleen cultures, offering support to the specificity of the approach and suggesting that 3c-targeted therapeutics may not be limited to delivery by inhalation and could be administered via other routes without influencing systemic immunity.

Although previous studies targeting IL-5 in asthma have been successful in reducing some features of the late-phase allergic response (25, 33, 34), serum IgE, a crucial mediator of the early-phase response, has remained elevated (31). Here we demonstrate a significant reduction in circulating IgE in βc−/− mice (FIG. 5, A). Both IL-3 and GM-CSF are important mediators of mast cell and basophil function in allergic disease and the attenuation of IgE may be a consequence of reduced function of these cell types. Thus inactivation of the βc receptor has the potential to influence both early- and late-phase asthmatic responses.

An amplification of serum antigen-specific IgGᵢ and diminished antigen-specific IgG₂ₐ levels has been correlated with polarization of T cell responses to a type 2 phenotype in allergic airways disease. Intriguingly, we have reported an attenuation of Th2 cytokine responses and disease parameters accompanied by an antigen-specific increase in IgG₁ and decrease in IgG₂ₐ levels (FIG. 5, B-C). This deregulation of B cell responses implies the existence of novel pathways for B cell signalling that may involve the β common receptor and emphasises that although a common correlate of asthma, isotype switching to IgGᵢ is not obligatorily involved in Th2 cytokine production and allergic disease.

In a previous study examining the immune response of βc−/− mice to parasite infection, the eosinophil response at days 7 and 14, representing the peak response in WT mice, is absent (47). However, when these researchers extended their analysis for a further period they discovered that in the absence of βc, some granulomatous lesions with infiltration of eosinophils were observed at day 21, when WT eosinophilia is normally resolved. For this reason it was important to conduct a temporal analysis of the allergic airways model in βc−/− mice to determine whether an eosinophil response is detected in these mice beyond 24 h (day 1) after final antigen challenge. βc−/− mice did not develop a delayed eosinophilic response to the allergic model in the blood, airway or pulmonary tissue (FIG. 6, A-C).

In the lung, inhaled antigens are captured by a network of dendritic cells (DCs) resident within the mucosa. Antigen recognition in the context of a danger signal is thought to be the basis for DC maturation and migration to the lung-draining lymph nodes, where antigen processing by the MHC II complex and subsequent interaction with the T cell receptor and various costimulatory molecules facilitates T cell activation and influences polarization of the effector cell response. Activated Th2 cells then migrate into the pulmonary tissue and secrete cytokines, contributing to the allergic airway response. Our observation of reduced Th2 cytokine production by peribronchial lymph nodes (FIG. 4) raises the question of whether βc−/− T cells have an intrinsic defect in the ability to produce cytokine, or alternatively whether the pulmonary microenvironment is altered in terms of the ability of DCs to activate T cells and promote their migration into the lung tissue.

CD4+ T cells isolated from βc−/− mice were found to have selective defects in the ability to produce cytokine after in vitro incubation under Th2-polarising conditions (FIG. 7). Although IL-5 production was comparable to the WT mouse, the production of IL-13, IL-4 and GM-CSF were attenuated. A decrease in IFN-γ was also perceptible, but these cells were prepared under conditions designed to promote Th2 differentiation, and the absolute levels are too low to be considered of any physiological significance. Thus although the fundamental ability of T cells from βc−/− mice to produce cytokine is somewhat diminished, this cannot entirely account for the striking reduction in cytokine production in the PBLN following application of the in vivo allergy model. Flow cytometry on cells recovered from the PBLN of allergic mice revealed that despite having a reduced functional capacity (FIG. 4), the PBLN of βc−/− mice actually harbours more CD4+ lymphocytes compared to WT controls (FIG. 8, A). This may be explained in part by decreased cell activation in this compartment (FIG. 8, B). Additionally, the virtual absence of lymphocytes in lung tissue from allergic βc−/− mice indicates further dysfunction in the migration from the draining lymph nodes into the lung following antigen provocation and the increased number of PBLN lymphocytes in βc−/− mice may be explained by a retention of cells (FIG. 8, C-D).

Defects in PBLN lymphocyte activation and migration invite speculation regarding the function dendritic cells in this compartment. We examined the phenotype and activation of myeloid (mDC) and plasmacytoid (pDC) dendritic cells in the PBLN and lung tissue of sensitised and challenged WT and βc−/− mice. DCs of the myeloid lineage have been demonstrated to play a key role in the response to inhaled antigen (54). βc−/− mice possess significantly fewer mDCs in the PBLN and lung following antigen challenge (FIG. 9, A-B). Further, although the expression of costimulatory markers by mDC in the PBLN does not appear to be remarkably influenced by βc function, activated mDC are virtually absent from the βc−/− lung (FIG. 8, B, D). It is tempting to speculate that the reduced activation of CD4+ T cells in the PBLN of βc−/− mice and attenuated migration into lung tissues may be attributable to ineffective antigen presentation and costimulation by mDCs. Although the role of pDCs remains unresolved in the current literature, these cells are generally thought to contribute largely to the establishment of tolerance and the immune response to viral infection (54). By contrast to mDCs, there are no differences in pDC numbers in the lymph node of allergic βc−/− mice compared to WT controls (FIG. 8, A). However the reduction in lung pDC levels in the absence of βc signalling raises important questions regarding the response of these mice to viral infection that warrant further investigation.

### REFERENCES

1. Beasley et al., (2000) J Allergy Clin Immunol 105:S466-472.
2. Braman (2006) Chest 130:4 S-12S.
3. Peat et al., (1994) Bmj 308:1591-1596.
4. Sly (2004). Ann Allergy Asthma Immunol 92:313-318.
5. Bochner et al., (1994) Annu Rev Immunol 12:295-335.
6. Djukanovic et al., (1990) Am Rev Respir Dis 142:434-457.
7. Lewkowich et al., (2005) J Exp Med 202:1549-1561.
8. Strickland et al., (2006) J Exp Med
9. Umetsu and DeKruyff. (2006) Immunol Rev 212:238-255.
10. Akbari et al., (2003) Nat Med 9:582-588.
11. Korsgren (2002) Curr Pharm Des 8:1871-1876.
12. Lisbonne et al., (2003) J Immunol 171:1637-1641.
13. Trottein et al., (2006) Chem Immunol Allergy 90:113-127.
14. Robinson et al., (1992) N Engl J Med 326:298-304.
15. Walker et al., (1992) Am Rev Respir Dis 146:109-115.
16. Wills-Karp (1999) Annu Rev Immunol 17:255-281.
17. De Monchy et al., (1985) Am Rev Respir Dis 131:373-376.
18. Gleich et al., (1988) J Allergy Clin Immunol 81:776-781.
19. Rothenberg and Hogan. (2006). The eosinophil. Annu Rev Immunol 24:147-174.
20. Dahl et al., (1978) Allergy 33:211-215.
21. Djukanovic et al., (1990) Am Rev Respir Dis 142:863-871.
22. Robinson et al., (1993) J Allergy Clin Immunol 92:397-403.
23. Walker et al., (1991)J Allergy Clin Immunol 88:935-942.
24. Bentley et al., (1993) Am J Respir Cell Mol Biol 8:35-42.
25. Foster et al., (1996) J Exp Med 183:195-201.
26. Hogan et al., (1998) Am J Respir Crit. Care Med 157:210-218.
27. Lopez et al., (1988) J Exp Med 167:219-224.
28. Sur et al., (1995) J Allergy Clin Immunol 96:661-668.
29. Lee et al., (1997) J Immunol 158:1332-1344.
30. Corry et al., (1996) JExp Med 183:109-117.
31. Hamelmann et al., (1997) Am JRespir Crit. Care Med 155:819-825.
32. Hogan et al., (1998) J Immunol 161:1501-1509.
33. Mauser et al., (1993) Am Rev Respir Dis 148:1623-1627.
34. Nagai et al., (1993). Life Sci 53:PL243-247.
35. Buttner et al., (2003). Eur Respir J21:799-803.
36. Kay and Klion. (2004). Immunol Allergy Clin North Am 24:645-666, vii.
37. Kips et al., (2003). Am JRespir Crit. Care Med 167:1655-1659.
38. Leckie et al., (2000). Lancet 356:2144-2148.
39. Begley et al., (1986). Blood 68:162-166.
40. Lopez et al., (1986). J Clin Invest 78:1220-1228.
41. Rothenberg et al., (1988). J Clin Invest 81:1986-1992.
42. Martinez-Moczygemba and Huston. (2003). J Allergy Clin Immunol 112:653-665; quiz 666.
43. Pazdrak et al., (1998). J Exp Med 188:421-429.
44. Simon et al., (1997). Eur J Immunol 27:3536-3539.
45. Yousefi et al., (1996). J Exp Med 183:1407-1414.
46. Robb et al., (1995). Proc Natl Acad Sci USA 92:9565-9569.
47. Nishinakamura et al. (1995). Immunity 2:211-222.
48. Sun et al., (1999). Blood 94:1943-1951.
49. Adachi et al., (1999). J Immunol 163:939-946.
50. Allakhverdi et al., (2002). Am JRespir Crit. Care Med 165:1015-1021.
51. Ikegami et al., (1996). Am J Physiol 270:L650-658.
52. Dranoff et al. (1994). I Science 264:713-716.
53. Stanley et al., 1994. Proc Natl Acad Sci USA 91:5592-5596.
54. Hammad and Lambrecht. (2006). J Allergy Clin Immunol 118:331-336.
55. Kopf et al., (1993). Nature 362:245.
56. Wills-Karp et al., (1998). Science 282:2258
57. Zhu et al., (1999). J. Clin. Invest. 103:779.
58. Dabbagh et al., (1999). J. Immunol. 162:6233.
59. De Vries. (1998). J. Allergy Clin. Immunol. 102:165.
60. Grunig et al., (1998). Science 282:2261.
61. Rossjohn et al., (2000). Blood 95:2491.

1.-18. (canceled)

19. The method of treatment of a severe inflammatory obstructive airway condition as in claim 39 wherein the asthma exhibits and the βc blocker is administered for a time sufficient to cause a reduction of lung remodelling.

20. The method of treatment of a severe inflammatory obstructive airway condition as in claim 19 including the step of estimating the degree of lung remodeling before administering βc blocker and estimating the degree of lung remodelling after administering βc blocker and assessing the degree of reduction of the lung remodelling.

21.-22. (canceled)

23. The method of treatment of a severe inflammatory obstructive airway condition as in claim 19 wherein a preferential reduction in the lung of one or more Th2 cytokine levels is achieved relative to systemic levels.

24. The method of treatment of a severe inflammatory obstructive airway condition as in claim 23 wherein the βc blocker is maintained in the lung of the mammal at an effective level for at least one week.

25. The method of treatment of a severe inflammatory obstructive airway condition as in claim 23 wherein the βc blocker is maintained in the lung of the mammal at an effective level for at least one month.

26. The method of treatment of a severe inflammatory obstructive airway condition as in claim 23 wherein the βc blocker is maintained in the lung of the mammal at an effective level for at least one year.

27. The method of treatment of a severe inflammatory obstructive airway condition as in claim 24 wherein the βc blocker is administered by slow or controlled release delivery.

28. The method of treatment of a severe inflammatory obstructive airway condition as in claim 24 wherein the βc blocker is administered two more times temporally spaced apart.

29. The method of treatment of a severe inflammatory obstructive airway condition as in claim 19 wherein the βc blocker is administered by non-pulmonary delivery.

30. The method of treatment of a severe inflammatory obstructive airway condition as in claim 29 wherein the βc blocker is administered transdermally or transmucosally.

31. The method of treatment of a severe inflammatory obstructive airway condition as in claim 30 wherein the βc blocker is administered in a slow release depot.

32. The method of treatment of a severe inflammatory obstructive airway condition as in claim 19 wherein the βc blocker comprises an antibody or a fragment thereof.

33. The method of treatment of a severe inflammatory obstructive airway condition as in claim 20 wherein the degree of lung remodelling is estimated by a method selected from the group consisting of (a) respiratory function measurement, (b) arthroscopic measurement of airway constriction, and (c) an extent of airway hypersensitive reaction on challenge with a provoking agent.

34. The method of treatment of a severe inflammatory obstructive airway condition as in claim 19 wherein the mammal has exhibited clinical manifestations of the obstructive airways condition.

35. The method of treatment of a severe inflammatory obstructive airway condition as in claim 19 wherein the obstructive airways condition is sub-clinical.

36. A method of treatment of a severe inflammatory obstructive airway condition in a mammal, the condition being refractory to treatment with glucocorticosteroids, the method comprising the step of administering one or more times a βc blocker capable of blocking the binding of all three of IL-3, IL-5 and GM-CSF to the common βc.

37. The method of treatment of a severe inflammatory obstructive airway condition as in claim 36, additionally including administering an additional active useful for alleviating the symptoms of at least non-severe cases of the inflammatory obstructive airway condition in the mammal.

38. The method of treatment of a severe inflammatory obstructive airway condition as in claim 37, said additional active being selected from the group consisting of glucocorticosteroids, beta-agonists and anticholinergic agents.

39. The method of treatment of a severe inflammatory obstructive airway condition as in claim 37 wherein the mammal is asthmatic.

40. The method of treatment of a severe inflammatory obstructive airway condition as in claim 37 wherein the βc blocker is administered in slow release form to be present at an effective level in the lung for at least a week and the additional active is administered during an attack.

41. The method of treatment of a severe inflammatory obstructive airway condition as in claim 40 wherein the βc blocker is administered during an attack.

42. The method of treatment of a severe inflammatory obstructive airway condition as in claim 37 wherein the βc blocker is administered together with the additional agent.

43. The method of treatment of a severe inflammatory obstructive airway condition as in claim 42 wherein administration of the additional agent and the βc blocker is pulmonary.

44.-83. (canceled)
